SEQUENCE LISTING (10 sequences)

SEQ ID NO: 1 - protein, 123 aa, synthetic construct
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 2 - protein, 107 aa, synthetic construct
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 3 - protein, 8 aa, synthetic construct
GFTFSSYG

SEQ ID NO: 4 - protein, 8 aa, synthetic construct
IWYDGSNK

SEQ ID NO: 5 - protein, 16 aa, synthetic construct
ASVATSGDFDYYGMDV

SEQ ID NO: 6 - protein, 6 aa, synthetic construct
QRISTY

SEQ ID NO: 7 - skipped

SEQ ID NO: 8 - protein, 9 aa, synthetic construct
QQRSNWPLT

SEQ ID NO: 9 - protein, 449 aa, synthetic construct
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0179177 A1
(43) Pub. Date: Jun. 5, 2025

(54) STABLE ANTIBODY FORMULATION

(71) Applicant: Regeneron Pharmaceuticals, Inc., Tarrytown, NY (US)

(72) Inventors: Xiaolin TANG, Old Tappan, NJ (US); Hunter H. CHEN, New York, NY (US); Mary KLEPPE, Milford, CT (US)

(21) Appl. No.: 18/930,913

(22) Filed: Oct. 29, 2024

Related U.S. Application Data
(60) Provisional application No. 63/594,383, filed on Oct. 30, 2023.
(60) Provisional application No. 63/700,580, filed on Sep. 27, 2024.

(51) Int. Cl. C07K 16/28; A61K 39/00; A61K 47/18; A61K 47/22; A61K 47/26

(52) U.S. Cl. CPC C07K 16/2803; A61K 47/183; A61K 47/22; A61K 47/26; A61K 2039/545; C07K 2317/565; C07K 2317/94

(57) ABSTRACT

The present invention provides stable pharmaceutical formulations comprising a human antibody that specifically binds to human lymphocyte activation gene-3 (LAG-3). In certain embodiments, the formulations contain, in addition to an anti-LAG-3 antibody, a buffer, an amino acid, a non-ionic surfactant, and a sugar. The pharmaceutical formulations of the present invention exhibit a substantial degree of antibody stability upon stress and storage.

### CROSS-REFERENCE TO RELATED APPLICATIONS

This application claims priority to U.S. Provisional Patent Application No. 63/700,580, filed Sep. 27, 2024, and U.S. Provisional Patent Application No. 63/594,383, filed Oct. 30, 2023, the contents of which are incorporated by reference herein.

### FIELD

The present disclosure relates to the field of therapeutic antibody formulations. More specifically, the present disclosure relates to the field of pharmaceutical formulations comprising a human antibody that specifically binds to human lymphocyte activation gene-3 (LAG-3) protein.

### SEQUENCE LISTING

An official copy of the sequence listing is submitted concurrently with the specification via Patent Center. The content of the electronic sequence listing (11396US01_Sequence_Listing_ST26.xml; Size: 12,288 bytes; and Date of Creation: Oct. 29, 2024) is part of the specification and is herein incorporated by reference in its entirety.

### BACKGROUND

Therapeutic macromolecules (e.g., antibodies) must be formulated in a manner that not only makes the molecules suitable for administration to patients, but also maintains their stability during storage and subsequent use. For example, therapeutic antibodies in liquid solution are prone to degradation, aggregation or undesired chemical modifications unless the solution is formulated properly. The stability of an antibody in liquid formulation depends not only on the kinds of excipients used in the formulation, but also on the amounts and proportions of the excipients relative to one another. Furthermore, other considerations aside from stability must be taken into account when preparing a liquid antibody formulation. Examples of such additional considerations include the viscosity of the solution and the concentration of antibody that can be accommodated by a given formulation, and the visual quality or appeal of the formulation. Thus, when formulating a therapeutic antibody, great care must be taken to arrive at a formulation that remains stable, contains an adequate concentration of antibody, and possesses a suitable viscosity as well as other properties which enable the formulation to be conveniently administered to patients.

Antibodies to the human lymphocyte activation gene-3 (LAG-3) are one example of a therapeutically relevant macromolecule that requires proper formulation. Anti-LAG-3 antibodies are clinically useful stimulating or enhancing the immune response and/or for treating a subject suffering from cancer, or a chronic viral infection. Exemplary anti-LAG3 antibodies useful herein include, inter alia, LAG525 (and other LAG3 antibodies disclosed in U.S. 20100233183), relatlimab (and other LAG3 antibodies disclosed in U.S. 20110150892), GSK2831781 (and other LAG3 antibodies disclosed in U.S. 20140286935), MGD013 (and other LAG3 antibodies disclosed in WO2015200119) and LAG3 antibodies disclosed in U.S. 20160222116, U.S. 20170022273, U.S. 20170097333, U.S. 20170137517, U.S. 20170267759, U.S. 20170290914, U.S. 20170334995, WO2017062888, WO2016126858, WO2016200782, WO2017087589, WO2017087901, WO2017106129, WO2017149143, WO2017198741, WO2017219995, and WO2017220569.

Although anti-LAG-3 antibodies are known, there remains a need in the art for novel pharmaceutical formulations comprising anti-LAG-3 antibodies that are sufficiently stable and suitable for administration to patients.

### BRIEF SUMMARY

The present disclosure satisfies the aforementioned need by providing stable pharmaceutical formulations comprising a human antibody that specifically binds to human lymphocyte activation gene-3 (LAG-3).

In one aspect, a stable liquid pharmaceutical formulation of low viscosity is provided, comprising: (i) a human antibody that specifically binds to human lymphocyte activation gene-3 protein (LAG-3); (ii) a buffer; (iii) an organic cosolvent; and (iv) a stabilizer.

In various embodiments, the antibody is provided at a concentration from about 5±0.75 mg/mL to about 250±45 mg/mL. In one embodiment, the antibody is provided at a concentration of 12.5 mg/mL±1.85 mg/mL, or about 12.5 mg/mL. In one embodiment, the antibody is provided at a concentration of 25 mg/mL±3.75 mg/mL, or about 25 mg/mL. In another embodiment, the antibody is provided at a concentration of 50 mg/mL±7.5 mg/mL, or about 50 mg/mL. In another embodiment, the antibody is provided at a concentration of 100 mg/mL±15 mg/mL, or about 100 mg/mL. In one embodiment, the antibody is provided at a concentration of 150 mg/mL±22.5 mg/mL, or about 150 mg/mL. In another embodiment, the antibody is provided at a concentration of 175 mg/mL±26.25 mg/mL, or about 175 mg/mL. In another embodiment, the antibody is provided at a concentration of 200 mg/mL±30 mg/mL, or about 200 mg/mL.

In certain embodiments, the formulation comprises any one of the anti-LAG-3 antibodies disclosed in U.S. 20170101472, incorporated herein in its entirety. In certain embodiments, the anti-LAG-3 antibody comprises (a) a heavy chain variable region (HCVR) comprising heavy chain complementarity determining regions 1, 2 and 3 (HCDR1-HCDR2-HCDR3) each comprising a sequence of SEQ ID NO: 3, SEQ ID NO: 4 and SEQ ID NO: 5, respectively; and (b) a light chain variable region (LCVR) comprising light chain complementarity determining regions 1, 2 and 3 (LCDR1-LCDR2-LCDR3) each comprising a sequence of SEQ ID NO: 6, SEQ ID NO: 7 and SEQ ID NO: 8, respectively. In one embodiment, the antibody comprises a HCVR comprising the amino acid sequence of SEQ ID NO: 1 and a LCVR comprising the amino acid sequence of SEQ ID NO: 2. In one embodiment, the antibody comprises a heavy chain comprising the amino acid sequence of SEQ ID NO: 9 and a light chain comprising the amino acid sequence of SEQ ID NO: 10. In one embodiment, the antibody comprises a HCVR having 90% sequence identity to SEQ ID NO: 1. In one embodiment, the antibody comprises a LCVR having 90% sequence identity to SEQ ID NO: 2. In one embodiment, the antibody comprises a HCVR having 90% sequence identity to SEQ ID NO: 1 and a LCVR having 90% sequence identity to SEQ ID NO: 2. In one embodiment, the antibody comprises a heavy chain and light chain, wherein the heavy chain comprises an amino acid sequence comprising amino acids 1-448 of SEQ ID NO: 9. In one embodiment, the antibody comprises a heavy chain and light chain, wherein the light chain comprises an amino acid sequence of SEQ ID NO: 10. In one embodiment, the antibody comprises a heavy chain/light chain comprising amino acid sequences of SEQ ID NOs: 9/10. In one embodiment, the antibody comprises a heavy chain amino acid sequence comprising amino acids 1-448 of SEQ ID NO: 9, and a light chain comprising an amino acid sequence of SEQ ID NO: 10.

In one embodiment, the pH of the liquid formulation is pH 6.0±0.5, pH 6.0±0.4, pH 6.0±0.3, pH 6.0±0.2, pH 6.0±0.1, pH 6.0±0.05, pH 6.0±0.01, or pH 6.0. In one embodiment, the pH of the liquid formulation is about pH 6.0±0.3.

In one embodiment, the buffer comprises histidine. In certain embodiments, the histidine buffer is at a concentration of from 5 mM±1 mM to 50 mM±10 mM, for example, from 5 mM±1 mM to 25 mM±5 mM. In one embodiment, the histidine buffer is at a concentration of 10 mM±2 mM or about 10 mM. In one embodiment, the histidine buffer is at a concentration of 20 mM±4 mM or about 20 mM. In one embodiment, the histidine buffer is at a concentration of 40 nM±8 mM or about 40 nM. In certain embodiments, the histidine buffer comprises L-histidine and L-histidine monohydrochloride monohydrate. In one embodiment, L-histidine is at a concentration of from 2 mM±0.4 mM to 25 mM±5 mM, for example, from 4 mM±0.8 mM to 20 mM±4 mM. In one embodiment, L-histidine monohydrochloride monohydrate is at a concentration of from 2 mM±0.4 mM to 25 mM±5 mM, for example, from 4 mM±0.8 mM to 20 mM±4 mM. In one embodiment, the buffer comprises L-histidine at a concentration of 5.0 mM±1.0 mM and L-histidine monohydrochloride monohydrate at a concentration of 5.0 mM±1.0 mM. In one embodiment, the buffer comprises histidine at a concentration of 10 mM±2 mM, wherein the histidine comprises L-histidine at a concentration of 5.0 mM±1.0 mM and L-histidine monohydrochloride monohydrate at a concentration of 5.0 mM±1.0 mM.

In certain embodiments, the organic cosolvent is a nonionic polymer containing a polyoxyethylene moiety. In one embodiment, the organic solvent is a surfactant. In some embodiments, the organic cosolvent is any one or more of polysorbate, poloxamer 188 and polyethylene glycol 3350. In one embodiment, the organic cosolvent is polysorbate 80. In one embodiment, the organic cosolvent is polysorbate 20. In one embodiment, the organic cosolvent is polyethylene glycol, e.g., PEG3350.

In one embodiment, the organic cosolvent is at a concentration of from about 0.01%±0.005% to about 1%±0.5% “weight to volume” or “w/v”, wherein, e.g., 0.1 g/ml=10% and 0.01 g/ml=1%. In certain embodiments, the organic solvent is polysorbate at a concentration of from 0.05%±0.025% to 0.5%±0.25% (w/v). In one embodiment, the organic cosolvent is polysorbate 80, which is at a concentration of 0.2%±0.02% w/v, or about 0.2%. In another embodiment, the organic cosolvent is polysorbate 80, which is at a concentration of 0.1%±0.05% w/v or about 0.1% w/v. In one embodiment, the organic cosolvent is polysorbate 20, which is at a concentration of 0.2%±0.1% w/v, or about 0.2%. In another embodiment, the organic cosolvent is polysorbate 20, which is at a concentration of 0.1%±0.05% w/v or about 0.1% w/v. In another embodiment, the organic cosolvent is PEG3350, which is at a concentration of 1.0%±0.5% w/v or about 1.0% w/v.

In certain embodiments, the stabilizer is a sugar. In one embodiment, the sugar is sucrose. In various embodiments, the stabilizer is at a concentration of from 1%±0.2% w/v to 20%±4% w/v, from 5%±1% w/v to 15%±3% w/v, or from 1%±0.2% to 10%±2% w/v. In one embodiment, the stabilizer is sucrose at a concentration of 5%±1% w/v or about 5% w/v. In another embodiment, the stabilizer is sucrose at a concentration of 9%±1.8% w/v or about 9% w/v. In another embodiment, the stabilizer is sucrose at a concentration of 10%±2% w/v or about 10% w/v.

In certain embodiments, the stabilizer is an amino acid. In one embodiment, the stabilizer is an amino acid such as arginine hydrochloride or proline. In one embodiment, the stabilizer is arginine hydrochloride. In certain embodiments, the stabilizer is arginine hydrochloride and is at a concentration of from 1 mM±2 mM to 100 mM±20 mM. In one embodiment, the stabilizer is arginine hydrochloride at a concentration of 20 mM±4 mM or about 20 mM. In one embodiment, the stabilizer is arginine hydrochloride at a concentration of 80 mM±16 mM or about 80 mM.

In certain embodiments, the stabilizer includes both sucrose and arginine. In another embodiment, the stabilizer is sucrose at a concentration of 10%±2% w/v or about 10% w/v and arginine hydrochloride at a concentration of 20 mM±4 mM or about 20 mM.

In one aspect, a stable liquid pharmaceutical formulation is provided, comprising: (i) from 5±0.75 mg/ml to 250±37.5 mg/ml of a human antibody that specifically binds to human LAG-3; (ii) from 0 mM to 40±8 mM histidine buffer; (iii) from 0% to 0.5%±0.25% (w/v) polysorbate 80; (iv) from 0% to 15%±3% (w/v) sucrose; and (ν) from 1 mM±0.2 mM to 100 mM±20 mM arginine hydrochloride, at a pH of from about 5.3 to about 6.7; wherein the anti-LAG-3 antibody comprises a heavy chain variable region (HCVR) and a light chain variable region (LCVR) such that the HCVR/LCVR combination comprises heavy and light chain complementarity determining regions (HCDR1-HCDR2-HCDR3/LCDR1-LCDR2-LCDR3), which comprise the amino acid sequences of SEQ ID NOs: 3-4-5/SEQ ID NOs: 6-7-8, respectively. In one embodiment, the anti-LAG-3 antibody comprises a heavy chain variable region (HCVR) and light chain variable region (LCVR) comprising an amino acid sequence of SEQ ID NO: 1 and SEQ ID NO: 2, respectively. In certain embodiments, the anti-LAG-3 antibody comprises a Fc region elected from the group consisting of human IgG1, IgG2, IgG3, and IgG4 isotypes. In one embodiment, the antibody comprises a human IgG4 isotype. In one embodiment, the antibody comprises a heavy chain comprising the amino acid sequence of SEQ ID NO: 9 and a light chain comprising the amino acid sequence of SEQ ID NO: 10. In one embodiment, the antibody has a molecular weight of 145 kDa±5 kDa.

In certain embodiments, a stable liquid pharmaceutical formulation is provided, comprising: (i) from 5±0.75 mg/ml to 250±37.5 mg/ml of a human antibody that specifically binds to human LAG-3; (ii) from 0 mM to 40±8 mM histidine buffer; (iii) from 0% to 0.5%±0.25% (w/v) polysorbate 80; (iv) from 0% to 15%±3% (w/v) sucrose; and (v) from 1 mM±0.2 mM to 100 mM±20 mM arginine hydrochloride, at a pH of from about 5.3 to about 6.7; wherein the anti-LAG-3 antibody comprises a HCVR and a LCVR, wherein the HCVR has 90% sequence identity to SEQ ID NO: 1 and/or the LCVR has 90% sequence identity to SEQ ID NO: 2. In one embodiment, the anti-LAG-3 antibody comprises a HCVR comprising the amino acid sequence of SEQ ID NO: 1 and a LCVR comprising the amino acid sequence of SEQ ID NO: 2. In one embodiment, the anti-LAG-3 antibody comprises a heavy chain comprising the amino acid sequence of SEQ ID NO: 9; and a light chain comprising the amino acid sequence of SEQ ID NO: 10.

In certain embodiments, a stable liquid pharmaceutical formulation is provided, comprising: (i) from 5±0.75 mg/ml to 250±37.5 mg/ml of a human antibody that specifically binds to human LAG-3; (ii) from 0 mM to 40±8 mM histidine buffer; (iii) from 0% to 0.5%±0.25% (w/v) polysorbate 80; (iv) from 0% to 15%±3% (w/v) sucrose; and (v) from 1 mM±0.2 mM to 100 mM±20 mM arginine hydrochloride, at a pH of from about 5.3 to about 6.7; wherein the anti-LAG-3 antibody comprises a HCVR and a LCVR, wherein the HCVR comprises an amino acid sequence of SEQ ID NO: 1 having no more than five amino acid substitutions, and wherein the LCVR comprises an amino acid sequence of SEQ ID NO: 2 having no more than two amino acid substitutions. In one embodiment, the anti-LAG-3 antibody comprises a HCVR comprising the amino acid sequence of SEQ ID NO: 1 and a LCVR comprising the amino acid sequence of SEQ ID NO: 2. In one embodiment, the anti-LAG-3 antibody comprises a heavy chain comprising the amino acid sequence of SEQ ID NO: 9; and a light chain comprising the amino acid sequence of SEQ ID NO: 10.

In certain embodiments, the formulation of any of the preceding aspects has an attribute selected from the group consisting of: (i) the formulation is stable to long-term storage at 5° C., as described herein; (ii) the formulation is stable to agitation stress as described herein; (iii) the formulation is stable even with up to ±50% variation in the formulation excipient concentrations, as described herein; (iv) the formulation is stable to and compatible with intravenous delivery devices and procedures; and (v) the formulation is stable to long-term storage in a glass vial.

In certain embodiments of this aspect, a stable liquid formulation is provided, comprising: (i) from 5±0.75 mg/ml to 250±37.5 mg/ml of a human antibody that specifically binds to human LAG-3; (ii) from 5 mM±1 mM to 20±4 mM histidine buffer; (iii) from 0.05%±0.025% to 0.3%±0.15% (w/v) polysorbate 80; (iv) from 1%±0.2% to 15%±3% (w/v) sucrose; and (v) from 10 mM±2 mM to 30 mM±6 mM arginine hydrochloride, at a pH of about 6.0, wherein the antibody comprises a HCVR/LCVR comprising an amino acid sequence pair of SEQ ID NOs: 1/2.

In one embodiment of this aspect, the stable liquid formulation comprises (i) 25±3.75 mg/mL of an anti-LAG-3 antibody; (ii) 10±2 mM histidine buffer; (iii) 0.1%±0.05% (w/v) polysorbate 80; (iv) from 20 mM±4 mM arginine hydrochloride; and (v) 5%±1% (w/v) sucrose, at a pH of 6.0±0.3, wherein the antibody comprises a HCVR/LCVR comprising an amino acid sequence pair of SEQ ID NOs: 1/2.

In one embodiment of this aspect, the stable liquid formulation comprises (i) 50±7.5 mg/mL of an anti-LAG-3 antibody; (ii) 10±2 mM histidine buffer; (iii) 0.1%±0.5% (w/v) polysorbate 80; (iv) 20 mM±4 mM arginine hydrochloride; and (v) 10%±2% (w/v) sucrose, at a pH of 6.0±0.3, wherein the antibody comprises a HCVR/LCVR comprising an amino acid sequence pair of SEQ ID NOs: 1/2. In one embodiment, more than 98% of the antibodies maintained purity upon storage for 12 months at 5° C.

In one embodiment of this aspect, the stable liquid formulation comprises (i) 50±7.5 mg/mL of an anti-LAG-3 antibody; (ii) 10±2 mM histidine buffer; (iii) 0.1%±0.5% (w/v) polysorbate 80; and (iv) 5%±1% (w/v) sucrose, at a pH of 6.0±0.3, wherein the antibody comprises a HCVR/LCVR comprising an amino acid sequence pair of SEQ ID NOs: 1/2.

In one embodiment, the stable liquid formulation comprises (i) 100±15 mg/mL of an anti-LAG-3 antibody; (ii) 10±2 mM histidine buffer; (iii) 0.1%±0.05% (w/v) polysorbate 80; (iv) 10 mM±2 mM to 100 mM±20 arginine hydrochloride; and (v) 1%±0.2% (w/v) sucrose, at a pH of 6.0±0.3, wherein the antibody comprises a HCVR/LCVR comprising an amino acid sequence pair of SEQ ID NOs: 1/2.

In one embodiment, the stable liquid formulation comprises (i) 150±22.5 mg/mL of an anti-LAG-3 antibody; (ii) 10±2 mM histidine buffer; (iii) 0.1%±0.05% (w/v) polysorbate 80; (iv) 1%±0.2% to 10%±2% (w/v) sucrose; and (v) from 1 mM±0.2 mM to 100 mM±20 mM arginine hydrochloride, at a pH of 6.0±0.3, wherein the antibody comprises a HCVR/LCVR comprising an amino acid sequence pair of SEQ ID NOs: 1/2.

In one embodiment of this aspect, the stable liquid formulation comprises (i) 175±26.25 mg/mL of an anti-LAG-3 antibody; (ii) 10±2 mM histidine buffer; (iii) 0.1%±0.05% (w/v) polysorbate 80; (iv) 1%±0.2% to 10%±2% (w/v) sucrose; and (v) 10 mM±2 mM to 100 mM±20 mM arginine hydrochloride, at a pH of 6.0±0.3, wherein the antibody comprises a HCVR/LCVR comprising an amino acid sequence pair of SEQ ID NOs: 1/2.

In one embodiment of this aspect, the stable liquid formulation comprises (i) 200±30.00 mg/mL of an anti-LAG-3 antibody; (ii) 10±2 mM histidine buffer; (iii) 0.1%±0.05% (w/v) polysorbate 80; (iv) 10%±2% (w/v) sucrose; and (v) 80 mM±16 mM arginine hydrochloride, at a pH of 6.0±0.3, wherein the antibody comprises a HCVR/LCVR comprising an amino acid sequence pair of SEQ ID NOs: 1/2.

In one embodiment, the stable liquid formulation comprises (a) 5-250 mg/ml of an antibody that binds specifically to LAG-3, wherein the antibody comprises an HCVR of SEQ ID NO: 1 and an LCVR of SEQ ID NO: 2, (b) 10 mM±2 mM histidine buffer, pH 6.0±0.3, (c) 0.1%±0.05% w/v polysorbate 80, and (d) 5%±1% w/v sucrose. In some aspects, the stable liquid formulation further comprises 10 mM±2 mM to 100 mM±20 mM arginine hydrochloride.

In one embodiment, after storage of the formulation at 400 for 28 days, 95% of the antibody is native and 45% of the antibody is of the main charge form. In one embodiment, after storage of the formulation at 25° for three months, ≥98% of the antibody is native and 52% of the antibody is of the main charge form. In one embodiment, after storage of the formulation at 5° for 12 months, >98% of the antibody is native and >50% of the antibody is of the main charge form. In one embodiment, more than 98% of the antibodies have native conformation upon storage for 24 months at 5° C. In one embodiment, at least 98% or more of the antibodies have native conformation after agitation for 120 minutes.

In one aspect, the present disclosure provides a stable liquid formulation comprising: (i) up to 100 mg/mL of an anti-LAG-3 antibody; (ii) from 2 mM±0.4 mM to 20 mM±4 mM histidine buffer; (iii) up to 20%±4% (w/v) sucrose; and (iv) up to 0.2%±0.1% w/v polysorbate, at pH 6.0±0.3. In one embodiment, the stable liquid formulation comprises 25 mg/mL of anti-LAG-3 antibody. In one embodiment, the stable liquid formulation comprises 50 mg/mL of anti-LAG-3 antibody. In one embodiment, the stable liquid formulation comprises 75 mg/mL of anti-LAG-3 antibody. In one embodiment, the stable liquid formulation comprises 10 mM±2 mM histidine buffer. In one embodiment, the stable liquid formulation comprises 5% sucrose. In one embodiment, the stable liquid formulation comprises 6% sucrose. In one embodiment, the stable liquid formulation comprises 9% sucrose. In one embodiment, the stable liquid formulation comprises 10% sucrose. In one embodiment, the stable liquid formulation comprises 0.1% polysorbate. In one embodiment, the stable liquid formulation comprises 0.2% polysorbate. In one embodiment, the polysorbate is polysorbate 80 or polysorbate 20. In one embodiment, the anti-LAG-3 antibody comprises a HCVR/LCVR of SEQ ID NOs: 1/2.

In one aspect, a stable liquid pharmaceutical formulation of any of the preceding aspects is provided in a container. In one embodiment, the container is a polycarbonate vial. In one embodiment, the container is a glass vial. In one embodiment, the vial is 2 ml, 5 ml, 10 mL, or 20 ml Type 1 clear glass vial. In one embodiment, the glass vial is a type 1 borosilicate glass vial with a fluorocarbon-coated butyl rubber stopper. In one embodiment, the container is a microinfuser. In one embodiment, the container is a syringe. In one embodiment, the container is a prefilled syringe. In one embodiment, the syringe comprises a fluorocarbon-coated plunger. In certain embodiments, the syringe is a 1 mL or 2.25 mL long glass syringe containing less than about 500 parts per billion of tungsten equipped with a 27-G needle, a fluorocarbon-coated butyl rubber stopper, and a latex-free, non-cytotoxic rubber tip cap. In one embodiment, the syringe is a 1 mL long glass syringe equipped with a 27-G thin wall needle, a FLUROTEC-coated 4023/50 rubber stopper, and a FM 27 rubber tip cap. In one embodiment, the syringe is a 1 mL, 2 mL, 3 mL, 5 mL, or 10 mL plastic syringe fitted with a needle.

In one aspect, a kit comprising a stable pharmaceutical composition of any one of the preceding aspects, a container, and instructions is provided. In one embodiment, the container is a glass vial. In one embodiment, the container is a prefilled syringe. In one embodiment, the syringe is a 1 mL or 2.25 mL long glass syringe equipped with a 27-G thin wall needle, a FLUROTEC-coated 4023/50 rubber stopper, and a FM 27 rubber tip cap. In one embodiment, the syringe is a 1 mL, 2 mL, 3 mL, 5 mL, or 10 mL plastic syringe fitted with a needle.

In certain embodiments, the present disclosure provides a prefilled syringe comprising a stable liquid pharmaceutical formulation comprising: (i) from 5±0.75 mg/ml to 250±37.5 mg/ml of a human antibody that specifically binds to human LAG-3; (ii) from 5 mM±1 mM to 20±4 mM histidine buffer; (iii) from 0.05%±0.025% to 0.3%±0.15% (w/v) polysorbate 80; (iv) from 1%±0.2% to 15%±3% (w/v) sucrose; and (v) from 0 to 100 mM±20 mM arginine hydrochloride, at a pH of 6.0±0.3, wherein the antibody comprises a HCVR/LCVR comprising an amino acid sequence pair of SEQ ID NOs: 1/2; wherein the formulation has an attribute selected from the group consisting of: (i) 98% of the antibody is in native form after storage at 5° C. for 24 months; (ii) 50% of the antibody is the main charge variant after storage at 5° C. for 24 months; (iii) 98% of the antibody is in native form after storage at 25° C. for 6 months; (iv) 45% of the antibody is the main charge variant after storage at 25° C. for 6 months; (v) over 90% of the antibodies have a molecular weight of 145 kDa±1 kDa; (vi) more than 98% of the antibodies have native conformation upon storage for 12 months at 5° C.; and (vii) at least 97% or more of the antibodies have native conformation upon storage at 40° C. for 28 days.

In certain embodiments the present disclosure provides a glass vial comprising a stable liquid pharmaceutical formulation comprising: (i) from 5±0.75 mg/ml to 250±37.5 mg/ml of a human antibody that specifically binds to human LAG-3; (ii) from 5 mM±1 mM to 20±4 mM histidine buffer; (iii) from 0.05%±0.025% to 0.3%±0.15% (w/v) polysorbate 80; (iv) from 1%±0.2% to 15%±3% (w/v) sucrose; and (v) from 0 to 100 mM±20 mM arginine hydrochloride, at a pH of 6.0±0.3, wherein the antibody comprises a HCVR/LCVR comprising an amino acid sequence pair of SEQ ID NOs: 1/2; wherein the formulation has an attribute selected from the group consisting of: (i) the formulation is stable to storage and stress in a glass vial; (ii) the formulation is stable to and compatible for use in IV delivery devices; (iii) the formulation is chemically and physically stable to dilution with standard diluents known in the art (e.g., 0.9% sodium chloride or 5% dextrose); (iv) the formulation is stable to IV bags made of glass or polymer plastics (e.g., polyvinyl chloride, phthalates, polyolefins or polypropylene); (v) the formulation is compatible with standard infusion pumps (e.g., peristaltic pump, fluid displacement pump); (vi) 90% of the antibodies have a molecular weight of 145 kDa±1 kDa; (vii) more than 98% of the antibodies have native conformation upon storage for 24 months at 5° C.; and (viii) at least 97% or more of the antibodies have native conformation upon storage at 25° C. for 6 months.

Other embodiments will become apparent from a review of the ensuing detailed description.

### BRIEF DESCRIPTION OF THE FIGURES

FIG. 1 shows the effect of polysorbate 80 concentration on the stability of 50 mg/mL mAb1 after agitation (48 hours of orbital shaker at 250 RPM).

FIG. 2 depicts effect on quality attributes following storage of the fourteen DP formulations (50 mg/mL; 800 mg DP) at 2-8° C. over a period of 12 months.

FIG. 3 depicts effect on quality attributes following storage of the fourteen DP formulations (50 mg/mL; 800 mg DP) at 25 C/60% RH over a period of 6 months.

FIG. 4 depicts effect of antibody concentration (mg/mL protein) on viscosity in formulations comprising 10 mM histidine, 5% sucrose, 70 mM arginine HCl, and 0.1% PS80, at pH 6.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about”, when used in reference to a particular recited numerical value or range of values, means that the value may vary from the recited value by no more than 1%. For example, as used herein, the expression “about 100” includes 99 and 101 and all values in between (e.g., 99.1, 99.2, 99.3, 99.4, etc.). Although any methods and materials similar or equivalent to those described herein can be used in the practice or testing of the present invention, preferred methods and materials are now described. All publications mentioned herein are incorporated herein by reference in their entirety.

As used herein, the expression “pharmaceutical formulation” means a combination of at least one active ingredient (e.g., a small molecule, macromolecule, compound, etc. which is capable of exerting a biological effect in a human or non-human animal), and at least one inactive ingredient which, when combined with the active ingredient or one or more additional inactive ingredients, is suitable for therapeutic administration to a human or non-human animal. The term “formulation”, as used herein, means “pharmaceutical formulation” unless specifically indicated otherwise. The present disclosure provides pharmaceutical formulations comprising at least one therapeutic polypeptide. According to certain embodiments of the present disclosure, the therapeutic polypeptide is an antibody, or an antigen-binding fragment thereof, which binds specifically to human lymphocyte activation gene-3 (LAG-3) protein. More specifically, the present disclosure includes pharmaceutical formulations that comprise: (i) a human antibody that specifically binds to human LAG-3 (ii) a histidine buffer; (iii) an organic cosolvent that is a non-ionic surfactant; (iv) a stabilizer that is a carbohydrate and/or an amino acid. Specific exemplary components and formulations included within the present invention are described in detail below.

Antibodies that Bind Specifically to Lag-3

The pharmaceutical formulations of the present disclosure may comprise a human antibody, or an antigen-binding fragment thereof, that binds specifically to human LAG-3. As used herein, the term “LAG-3” means human lymphocyte activation gene-3. Antibodies to human LAG-3 are described in, for example, U.S. 20100233183, U.S. 20110150892, U.S. 20140286935, WO2015200119, U.S. 20160222116, U.S. 20170022273, U.S. 20170097333, U.S. 20170137517, U.S. 20170267759, U.S. 20170290914, U.S. 20170334995, WO2017062888, WO2016126858, WO2016200782, WO2017087589, WO2017087901, WO2017106129, WO2017149143, WO2017198741, WO2017219995, and WO2017220569.

The term “antibody”, as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody”. Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementary determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Unless specifically indicated otherwise, the term “antibody”, as used herein, shall be understood to encompass complete antibody molecules as well as antigen-binding fragments thereof. The term “antigen-binding portion” or “antigen-binding fragment” of an antibody (or simply “antibody portion” or “antibody fragment”), as used herein, refers to one or more fragments of an antibody that retain the ability to specifically bind to human LAG-3 or an epitope thereof.

An “isolated antibody”, as used herein, is intended to refer to an antibody that is substantially free of other antibodies having different antigenic specificities (e.g., an isolated antibody that specifically binds human LAG-3 is substantially free of antibodies that specifically bind antigens other than human LAG-3).

The term “specifically binds”, or the like, means that an antibody or antigen-binding fragment thereof forms a complex with an antigen that is relatively stable under physiologic conditions. Specific binding can be characterized by a dissociation constant of at least about 1×10⁻⁸ M or greater. Methods for determining whether two molecules specifically bind are well known in the art and include, for example, equilibrium dialysis, surface plasmon resonance, and the like. An isolated antibody that specifically binds human LAG-3 may, however, have cross-reactivity to other antigens, such as LAG-3 molecules from other species (orthologs). In the context of the present disclosure, multispecific (e.g., bispecific) antibodies that bind to human LAG-3 as well as one or more additional antigens are deemed to “specifically bind” human LAG-3. Moreover, an isolated antibody may be substantially free of other cellular material or chemicals.

Exemplary anti-human LAG-3 antibodies that may be included in the pharmaceutical formulations of the present disclosure are set forth in patent application publications U.S. 20170101472 and WO2017062888, the disclosures of which are incorporated by reference in their entirety.

According to certain embodiments of the present disclosure, the anti-human LAG-3 antibody, or antigen-binding fragment thereof, comprises a heavy chain complementary determining region (HCDR) 1 of SEQ ID NO: 3, an HCDR2 of SEQ ID NO: 4, and an HCDR3 of SEQ ID NO: 5. In certain embodiments, the anti-human LAG-3 antibody, or antigen-binding fragment thereof, comprises an HCVR of SEQ ID NO: 1.

According to certain embodiments of the present disclosure, the anti-human LAG-3, or antigen-binding fragment thereof, comprises a light chain complementary determining region (LCDR) 1 of SEQ ID NO: 6, an LCDR2 of SEQ ID NO: 7, and an LCDR3 of SEQ ID NO: 8. In certain embodiments, the anti-human LAG-3 antibody, or antigen-binding fragment thereof, comprises an LCVR of SEQ ID NO: 2.

According to certain embodiments of the present disclosure, the anti-human LAG-3, or antigen-binding fragment thereof, comprises a HCVR having 90%, 95%, 98% or 99% sequence identity to SEQ ID NO: 1.

According to certain embodiments of the present disclosure, the anti-human LAG-3, or antigen-binding fragment thereof, comprises a LCVR having 90%, 95%, 98% or 99% sequence identity to SEQ ID NO: 2.

According to certain embodiments of the present disclosure, the anti-human LAG-3, or antigen-binding fragment thereof, comprises a HCVR comprising an amino acid sequence of SEQ ID NO: 1 having no more than 5 amino acid substitutions.

According to certain embodiments of the present disclosure, the anti-human LAG-3, or antigen-binding fragment thereof, comprises a LCVR comprising an amino acid sequence of SEQ ID NO: 2 having no more than 2 amino acid substitutions.

Sequence identity may be measured by any method known in the art (e.g., GAP, BESTFIT, and BLAST).

The present disclosure also includes formulations comprising anti-LAG-3 antibodies, wherein the anti-LAG-3 antibodies comprise variants of any of the HCVR, LCVR and/or CDR amino acid sequences disclosed herein having one or more conservative amino acid substitutions. For example, the present disclosure includes formulations comprising anti-LAG-3 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

In certain embodiments, the anti-LAG-3 antibody comprises a Fc region elected from the group consisting of human IgG1, IgG2, IgG3, and IgG4 isotypes.

The non-limiting, exemplary antibody used in the Examples herein is referred to as “mAb1”. This antibody is also referred to in U.S. 20170101472 as H4sH15482P, and is also known as “REGN3767” or “Fianlimab”. mAb1 (H4sH15482P) comprises an HCVR/LCVR amino acid sequence pair having SEQ ID NOs: 1/2, and HCDR1-HCDR2-HCDR3/LCDR1-LCDR2-LCDR3 domains represented by SEQ ID NOs: 3-4-5/SEQ ID NOs: 6-7-8.

According to certain embodiments of the present disclosure, the anti-human LAG-3, or antigen-binding fragment thereof, comprises a heavy chain of SEQ ID NO: 9 and a light chain of SEQ ID NO: 10.

It is well known in the art that terminal cleavage of amino acids can occur during production of antibodies (see, for example, Wang et al 2007, J. Pharma. Sci. 96: 1-26). Accordingly, in certain embodiments, the anti-LAG-3 antibody comprises a heavy chain and a light chain, wherein the heavy chain comprises the amino acid sequence of SEQ ID NO: 9, wherein the C-terminal lysine is absent from the amino acid sequence of SEQ ID NO: 9. In certain embodiments, formulations of the present disclosure contain about 50%, about 60%, about 70%, about 80%, about 90%, about 95%, about 98% or more of the anti-LAG-3 antibody wherein the C-terminal lysine is absent.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used. In certain embodiments, the pharmaceutical formulations are liquid formulations that may contain 5±0.75 mg/mL to 250±37.5 mg/mL of antibody; 10±1.5 mg/mL to 240±36 mg/mL of antibody; 20±3.0 mg/mL to 230±34.5 mg/mL of antibody; 25±3.75 mg/mL to 240±36 mg/mL of antibody; 50±7.5 mg/mL to 230±34.5 mg/mL of antibody; 60±9 mg/mL to 240±36 mg/mL of antibody; 70±10.5 mg/mL to 230±34.5 mg/mL of antibody; 80±12 mg/mL to 220±33 mg/mL of antibody; 90±13.5 mg/mL to 210±31.5 mg/mL of antibody; 100±15 mg/mL to 200±30 mg/mL of antibody; 110±16.5 mg/mL to 190±28.5 mg/mL of antibody; 120±18 mg/mL to 180±27 mg/mL of antibody; 130±19.5 mg/mL to 170±25.5 mg/mL of antibody; 140±21 mg/mL to 160±24 mg/mL of antibody; 150±22.5 mg/mL of antibody; or 175±26.25 mg/ml. For example, the formulations of the present disclosure may comprise about 5 mg/mL; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 100 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; about 200 mg/mL; about 205 mg/mL; about 210 mg/mL; about 215 mg/mL; about 220 mg/mL; about 225 mg/mL; about 230 mg/mL; about 235 mg/mL; about 240 mg/mL; about 245 mg/mL; or about 250 mg/mL of an antibody or an antigen-binding fragment thereof, that binds specifically to human LAG-3.

### Excipients and pH

The pharmaceutical formulations of the present disclosure comprise one or more excipients. The term “excipient”, as used herein, means any non-therapeutic agent added to the formulation to provide a desired consistency, viscosity or stabilizing effect.

In certain embodiments, the pharmaceutical formulation of the invention comprises at least one organic cosolvent in a type and in an amount that stabilizes the human LAG-3 antibody under conditions of rough handling or agitation, such as, e.g., vortexing. In some embodiments, what is meant by “stabilizes” is the prevention of the formation of more than 3% aggregated antibody of the total amount of antibody (on a molar basis) over the course of rough handling. In some embodiments, rough handling is vortexing a solution containing the antibody and the organic cosolvent for about 60 minutes or about 120 minutes.

In certain embodiments, the organic cosolvent is a non-ionic surfactant, such as an alkyl poly(ethylene oxide). Specific non-ionic surfactants that can be included in the formulations of the present disclosure include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 181, poloxamer 188, poloxamer 407; or polyethylene glycol (PEG, e.g., PEG3350). Polysorbate 20 is also known as TWEEN 20, sorbitan monolaurate and polyoxyethylene sorbitan monolaurate. Poloxamer 188 is also known as PLURONIC F68.

The amount of non-ionic surfactant contained within the pharmaceutical formulations of the present disclosure may vary depending on the specific properties desired of the formulations, as well as the particular circumstances and purposes for which the formulations are intended to be used. In certain embodiments, the formulations may contain 0.01%±0.005% to 0.5%±0.25% surfactant. For example, the formulations of the present disclosure may comprise about 0.005%; about 0.01%; about 0.02%; about 0.03%; about 0.04%; about 0.05%; about 0.06%; about 0.07%; about 0.08%; about 0.09%; about 0.1%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; about 0.30%; about 0.35%; about 0.40%; about 0.45%; about 0.46%; about 0.47%; about 0.48%; about 0.49%; about 0.50%; about 0.55%; or about 0.60% polysorbate 20, polysorbate 80, or PEG3350.

The pharmaceutical formulations of the present disclosure may also comprise one or more stabilizers in a type and in an amount that stabilizes the human LAG-3 antibody under conditions of thermal stress or agitation stress. In some embodiments, what is meant by “stabilizes” is maintaining greater than about 91% of the antibody in a native conformation when the solution containing the antibody and the thermal stabilizer is kept at about 45° C. for up to about 28 days. In some embodiments, what is meant by “stabilizes” is wherein less than about 6% of the antibody is aggregated when the solution containing the antibody and the thermal stabilizer is kept at about 45° C. for up to about 28 days. As used herein, “native” means the major form of the antibody by size exclusion, which is generally an intact monomer of the antibody. The term “native” also refers to non-aggregated and non-degraded form of the antibody.

In certain embodiments, the thermal stabilizer is a sugar such as sucrose, the amount of which contained within the formulation can vary depending on the specific circumstances and intended purposes for which the formulation is used. In certain embodiments, the formulations may contain about 1% to about 15% sugar; about 2% to about 14% sugar; about 3% to about 13% sugar; about 4% to about 12% sugar; about 5% to about 12% sugar; about 6% to about 11% sugar; about 7% to about 10% sugar; about 8% to about 11% sugar; or about 9% to about 11% sugar. For example, the pharmaceutical formulations of the present disclosure may comprise 4%±0.8%; 5%±1%; 6%±1.2%; 7%±1.4%; 8%±1.6%; 9%±1.8%; 10%±2%; 11%±2.2%; 12%±2.4%; 13%±2.6%; about 14%±2.8% sugar; or about 15%±3.0% sugar (e.g., sucrose).

In certain embodiments, the stabilizer is an amino acid, for example, arginine. In certain embodiments, the formulations may contain 1-20% w/v arginine. In certain embodiments, the amino acid in the formulation may also act as a viscosity reducer, for example, in high-concentration formulations, e.g., when the concentration of the antibody is 100 mg/ml or more than 100 mg/ml. In some embodiments, when the concentration of the antibody is 175 mg/mL, arginine hydrochloride is present in the composition at a concentration from about 1 mM±0.2 mM to about 100 mM±20 mM and acts as a viscosity reducer. In some embodiments, the pharmaceutical formulation comprises 175 mg/mL anti-LAG3 antibody and 20 mM arginine hydrochloride.

The pharmaceutical formulations of the present disclosure may also comprise a buffer or buffer system, which serves to maintain a stable pH and to help stabilize the human LAG-3 antibody. The term “buffer” as used herein denotes a pharmaceutically acceptable buffer which maintains a stable pH or resists changes in pH of the solution. In some aspects, the buffer comprises phosphate. In some aspects, the buffer comprises histidine.

In the context of this disclosure, “histidine buffer” or “buffer comprising histidine” is a buffer comprising the amino acid histidine. Examples of histidine buffers include histidine chloride, histidine acetate, histidine phosphate, and histidine sulfate. In a one embodiment, the histidine buffer is prepared by dissolving L-histidine and L-histidine hydrochloride (e.g., as monohydrate) in a defined amount and ratio. In one embodiment, the histidine buffer is prepared by titrating L-histidine (free base, solid) with diluted hydrochloric acid. The term “histidine” is used interchangeably with “histidine buffer” throughout this disclosure. In some embodiments, what is meant by “stabilizes” is wherein less than 4.5%±0.5% of the antibody is aggregated when the solution containing the antibody and the buffer is kept at about 40° C. for up to about 28 days.

In some embodiments, what is meant by “stabilizes” is wherein less than 2%±0.5% or less than 1%±0.5% of the antibody is aggregated when the solution containing the antibody and the buffer is kept at about 40° C. for up to about 28 days. In some embodiments, what is meant by “stabilizes” is wherein at least 97%±0.5% or at least 98%±0.5% of the antibody is in its native conformation as determined by size exclusion chromatography when the solution containing the antibody and the buffer is kept at about 40° C. for up to about 28 days. By “native” or “native conformation”, what is meant is the antibody fraction that is not aggregated or degraded. This is generally determined by an assay that measures the relative size of the antibody entity, such as a size exclusion chromatographic assay. The non-aggregated and non-degraded antibody elutes at a fraction that equates to the native antibody, and is generally the main elution fraction.

Aggregated antibody elutes at a fraction that indicates a size greater than the native antibody. Degraded antibody elutes at a fraction that indicates a size less than the native antibody.

In some embodiments, what is meant by “stabilizes” is wherein at least 35%±0.5% of the antibody is in its main charge form as determined by cation exchange chromatography when the solution containing the antibody and the buffer is kept at about 40° C. for up to about 28 days. In some embodiments, what is meant by “stabilizes” is wherein at least 46%±0.5% of the antibody is in its main charge form as determined by cation exchange chromatography when the solution containing the antibody and the buffer is kept at about 40° C. for up to about 28 days. By “main charge” or “main charge form”, what is meant is the fraction of antibody that elutes from an ion exchange resin in the main peak, which is generally flanked by more “basic” peaks on one side and more “acidic” peaks on the other side.

The pharmaceutical formulations of the present disclosure may have a pH of from about 5.2 to about 6.4. For example, the formulations of the present disclosure may have a pH of about 5.5; about 5.6; about 5.7; about 5.8; about 5.9; about 6.0; about 6.1; about 6.2; about 6.3; about 6.4; or about 6.5. In some embodiments, the pH is 6.0±0.4; 6.0±0.3; 6.0±0.2; 6.0±0.1; about 6.0; or 6.0.

In some embodiments, the buffer or buffer system comprises at least one buffer that has a buffering range that overlaps fully or in part the range of pH 5.5-7.4. In certain embodiments, the buffer comprises a histidine buffer. In certain embodiments, the histidine buffer is present at a concentration of 5 mM±1 mM to 15 mM±3 mM; 6 mM±1.2 mM to 14 mM±2.8 mM; 7 mM±1.4 mM to 13 mM±2.6 mM; 8 mM±1.6 mM to 12 mM±2.4 mM; 9 mM±1.8 mM to 11 mM±2.2 mM; 10 mM±2 mM; or about 10 mM. In certain embodiments, the buffer system comprises histidine at 10 mM±2 mM, at a pH of 6.0±0.3. In certain embodiments, the histidine buffer comprises L-histidine and L-histidine monohydrochloride monohydrate. In one embodiment, the histidine buffer comprises L-histidine at a concentration of 5.0 mM±1.0 mM. In one embodiment, the histidine buffer comprises L-histidine monohydrochloride monohydrate at a concentration of 5.0 mM±1.0 mM. In one embodiment, the histidine buffer comprises L-histidine at a concentration of 5.0 mM±1.0 mM and L-histidine monohydrochloride monohydrate at a concentration of 5.0 mM±1.0 mM.

The pharmaceutical formulations of the present disclosure may also comprise one or more excipients that serve to maintain a reduced viscosity or to lower the viscosity of formulations containing of anti-LAG-3 antibody drug substance (e.g., 50 mg/ml of antibody), and in some aspects, a high concentration of LAG-3 antibody drug substance (e.g., generally 150 mg/ml of antibody). In certain embodiments, the stabilizer is an amino acid. In one embodiment, the amino acid is arginine hydrochloride. In one embodiment, the pharmaceutical formulation of the present disclosure contains arginine hydrochloride at a concentration of 1 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, or 90 mM. In some embodiments, the formulation comprises arginine hydrochloride in an amount sufficient to maintain the viscosity of the liquid formulation at less than 20±3 cPoise, less than 15±2.25 cPoise, or less than 11±1.65 cPoise. In some embodiments, the formulation comprises arginine hydrochloride in an amount sufficient to maintain the viscosity at or below 15±2.25 cPoise. In certain embodiments, formulations may contain about 1 mM to about 80 mM; about 10 mM to about 30 mM arginine hydrochloride; or about 20 mM arginine hydrochloride. For example, the pharmaceutical formulations of the present disclosure may comprise 1 mM±0.2 mM, 10 mM±2 mM, 15 mM±3 mM, 20 mM±4 mM, 25 mM±5 mM, 30 mM±6 mM, 35 mM±7 mM, 40 mM±8 mM, 45 mM±9 mM, 50 mM±10 mM, 55 mM 11 mM, 60 mM±12 mM, 65 mM±13 mM, 70 mM±14 mM, 75 mM±15 mM, 80 mM±16 mM, 85 mM±17 mM, or 90 mM±18 mM arginine hydrochloride.

During the antibody purification process, it may be desired or necessary to exchange one buffer for another to achieve appropriate excipient concentrations, antibody concentration, pH, etc. Buffer exchange can be accomplished, e.g., by ultrafiltration/diafiltration (UF/DF) using, e.g., a semi-permeable tangential flow filtration membrane. Use of such techniques, however, has the potential to cause the Gibbs-Donnan effect [Bolton et al., 2011, Biotechnol. Prog. 27(1):140-152]. The buildup of positive charge on the product side of the membrane during protein concentration is counterbalanced electrically by the preferential movement of positive ions to the opposite side of the membrane. The potential consequence of this phenomenon is that the final concentrations of certain components (e.g., arginine hydrochloride, histidine, L-proline, etc.) may be lower than the intended target concentrations of these components due to the electrostatic repulsion of positively charged diafiltration buffer excipients to the positively charged antibody protein during the UF/DF step. Thus, the present disclosure includes formulations in which the concentration of, e.g., histidine and/or arginine hydrochloride vary from the recited amounts or ranges herein due to the Gibbs-Donnan effect.

Volume exclusion describes the behavior of highly concentrated samples in which a significant portion of the total volume of the solution is taken up by the solute, especially large molecules such as proteins, excluding the solvent from this space. This then decreases the total volume of solvent available for other solutes to be dissolved in, which may result in unequal partition across the ultrafiltration membrane. Thus, the present disclosure includes formulations in which the concentration of, e.g., histidine and/or arginine hydrochloride may vary from the recited amounts or ranges herein due to the volume exclusion effect.

During the manufacture of the formulations of the present disclosure, variations in the composition of the formulation may occur. These variations may include the concentration of the active ingredient, the concentration of the excipients, and/or the pH of the formulation. Because changes in any of these parameters could potentially impact the stability or potency of the drug product, proven acceptable range (PAR) studies were conducted to assess whether variations in the composition, within the defined ranges, would impact the stability or potency of the antibody. Accordingly, the present disclosure includes formulations comprising anti-LAG-3 antibodies which are stable and retain potency with up to 50% variation in the excipient concentration. For example, included herein are anti-LAG-3 antibody formulations, wherein stability and potency of said formulations is unaffected by ±10%, ±20%, ±30%, ±40% or ±50% variation in the concentration of antibody, sucrose, histidine buffer and/or polysorbate.

### Stability and Viscosity of the Pharmaceutical Formulations

The pharmaceutical formulations of the present disclosure typically exhibit high levels of stability. The term “stable”, as used herein in reference to the pharmaceutical formulations, means that the antibodies within the pharmaceutical formulations retain an acceptable degree of chemical structure or biological function after storage under defined conditions. A formulation may be stable even though the antibody contained therein does not maintain 100% of its chemical structure or biological function after storage for a defined amount of time. Under certain circumstances, maintenance of about 90%, about 95%, about 96%, about 97%, about 98% or about 99% of an antibody's structure or function after storage for a defined amount of time may be regarded as “stable”.

Stability can be measured, inter alia, by determining the percentage of native antibody that remains in the formulation after storage for a defined amount of time at a defined temperature. The percentage of native antibody can be determined by, inter alia, size exclusion chromatography (e.g., size exclusion ultra performance liquid chromatography [SE-UPLC]), such that native means non-aggregated and non-degraded. An “acceptable degree of stability”, as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a defined temperature. The defined amount of time after which stability is measured can be at least 14 days, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The defined temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C., or about 45° C. For example, a pharmaceutical formulation may be deemed stable if after 6 months of storage at 5° C., greater than about 95%, 96%, 97% or 98% of native antibody is detected by SE-UPLC. A pharmaceutical formulation may also be deemed stable if after 6 months of storage at 25° C., greater than about 95%, 96%, 97% or 98% of native antibody is detected by SE-UPLC. A pharmaceutical formulation may also be deemed stable if after 28 days of storage at 40° C., greater than about 89%, 90%, 91%, 92%, 93%, 94%, 95%, 96%, 97%, or 98% of native antibody is detected by SE-UPLC. A pharmaceutical formulation may also be deemed stable if after 12 months of storage at −20° C., greater than about 96%, 97%, or 98% of native antibody is detected by SE-UPLC. A pharmaceutical formulation may also be deemed stable if after 12 months of storage at −30° C., greater than about 96%, 97% or 98% of native antibody is detected by SE-UPLC. A pharmaceutical formulation may also be deemed stable if after 12 months of storage at −80° C., greater than about 96%, 97% or 98% of native antibody is detected by SE-UPLC.

Stability can be measured, inter alia, by determining the percentage of antibody that forms in an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion ultra performance liquid chromatography [SE-UPLC]). An “acceptable degree of stability”, as that phrase is used herein, means that at most 5% of the antibody is in an aggregated form (also denoted as the high molecular weight—HMW—form) detected in the formulation after storage for a defined amount of time at a given temperature. In certain embodiments an acceptable degree of stability means that at most about 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C. or about 45° C. For example, a pharmaceutical formulation may be deemed stable if after 12 months of storage at 5° C., less than about 2%, 1%, 0.5%, or 0.1% of the antibody is detected in an aggregated form. A pharmaceutical formulation may also be deemed stable if after three months of storage at 25° C., less than about 4%, 3%, 2%, 1%, 0.5%, or 0.1% of the antibody is detected in an aggregated form. A pharmaceutical formulation may also be deemed stable if after 28 days of storage at 45° C., less than about 10%, 9%, 8%, 7%, 6%, 5%, 4%, 3%, 2%, 1%, or 0.5%, of the antibody is detected in an aggregated form. A pharmaceutical formulation may also be deemed stable if after three months of storage at −20° C., −30° C., or −80° C. less than about 3%, 2%, 1%, 0.5%, or 0.1% of the antibody is detected in an aggregated form.

Stability can be measured, inter alia, by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99(8):5283-5288). The percentage of “acidified” antibody can be determined by, inter alia, ion exchange chromatography (e.g., cation exchange ultra performance liquid chromatography [CEX-UPLC]). An “acceptable degree of stability”, as that phrase is used herein, means that at most 45% of the antibody is in a more acidic form detected in the formulation after storage for a defined amount of time at a defined temperature. In certain embodiments an acceptable degree of stability means that at most about 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. In one embodiment, an acceptable degree of stability means that less than 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., or about 45° C. For example, a pharmaceutical formulation may be deemed stable if after three months of storage at −80° C., −30° C., or −20° C. less than about 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after six months of storage at 5° C., less than about 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after six months of storage at 25° C., less than about 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 45° C., less than about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody can be detected in a more acidic form.

Other methods may be used to assess the stability of the formulations of the present disclosure such as, e.g., differential scanning calorimetry (DSC) to determine thermal stability, controlled agitation to determine mechanical stability, and absorbance at about 350 nm or about 405 nm to determine solution turbidities. For example, a formulation of the present disclosure may be considered stable if, after 6 or more months of storage at about 5° C. to about 25° C., the change in OD₄₀₅ of the formulation is less than about 0.05 (e.g., 0.04, 0.03, 0.02, 0.01, or less) from the OD₄₀₅ of the formulation at time zero.

Measuring the biological activity or binding affinity of the antibody to its target may also be used to assess stability. For example, a formulation of the present disclosure may be regarded as stable if, after storage at e.g., 5° C., 25° C., 45° C., etc. for a defined amount of time (e.g., 1 to 12 months), the anti-LAG-3 antibody contained within the formulation binds to LAG-3 with an affinity that is at least 90%, 95%, or more of the binding affinity of the antibody prior to said storage. Binding affinity may be determined by e.g., ELISA or surface plasmon resonance. Biological activity may be determined by a LAG-3 activity assay, such as e.g., contacting a cell that expresses LAG-3 with the formulation comprising the anti-LAG-3 antibody. The binding of the antibody to such a cell may be measured directly, such as e.g., via FACS analysis. Alternatively, the downstream activity of the LAG-3 system may be measured in the presence of the antibody, and compared to the activity of the LAG-3 system in the absence of antibody. In some embodiments, the LAG-3 may be endogenous to the cell. In other embodiments, the LAG-3 may be ectopically expressed in the cell.

Additional methods for assessing the stability of an antibody in formulation are demonstrated in the Examples presented below.

The liquid pharmaceutical formulations of the present disclosure may, in certain embodiments, exhibit low to moderate levels of viscosity. “Viscosity” as used herein may be “kinematic viscosity” or “absolute viscosity”. “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. For example, if one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density (Absolute Viscosity=Kinematic Viscosity×Density). The dimension of kinematic viscosity is L²/T where L is a length and T is a time. Commonly, kinematic viscosity is expressed in centistokes (cSt). The SI unit of kinematic viscosity is mm²/s, which is 1 cSt. Absolute viscosity is expressed in units of centipoise (cP). The SI unit of absolute viscosity is the milliPascal-second (mPa-s), where 1 cP=1 mPa-s.

As used herein, a low level of viscosity, in reference to a fluid formulation of the present disclosure, will exhibit an absolute viscosity of less than about 20 cPoise (cP). For example, a fluid formulation of the invention will be deemed to have “low viscosity”, if, when measured using standard viscosity measurement techniques, the formulation exhibits an absolute viscosity of about 20 cP, about 19 cP, about 18 cP, about 15 cP, about 12 cP, about 10 cP, about 9 cP, about 8 cP, or less. As used herein, a moderate level of viscosity, in reference to a fluid formulation of the present disclosure, will exhibit an absolute viscosity of between about 35 cP and about 20 cP. For example, a fluid formulation of the invention will be deemed to have “moderate viscosity”, if when measured using standard viscosity measurement techniques, the formulation exhibits an absolute viscosity of about 34 cP, about 33 cP, about 32 cP, about 31 cP, about 30 cP, about 29 cP, about 28 cP, about 27 cP, about 26 cP, about 25 cP, about 24 cP, about 23 cP, about 22 cP, about 21 cP, about 20 cP, about 19 cP, 18 cP, about 17 cP, about 16 cP, or about 15 cP.

As illustrated in the examples below, the present inventors have made the surprising discovery that stable liquid formulations comprising high concentrations of an anti-human LAG-3 antibody (e.g., from about 50 mg/ml up to 250 mg/mL) can be obtained by formulating the antibody with arginine hydrochloride from about 1 mM to about 100 mM and sucrose at about 5% or about 10%. Such formulations are stable to stress during handling and to storage at temperatures ranging from 5° C. to 45° C. (shown herein).

### Exemplary Formulations

According to one aspect of the present disclosure, the pharmaceutical formulation is a stable, generally physiologically isotonic liquid formulation, which comprises: (i) a human antibody that specifically binds to human LAG-3 (e.g., REGN3767), at a concentration of up to 250 mg/mL±45 mg/mL; (ii) a histidine buffer system that provides sufficient buffering at about pH 6.0±0.3; (iii) an organic cosolvent, which protects the structural integrity of the antibody; (iv) a thermal stabilizer that is a sugar; and optionally, (v) an amino acid, which serves to retain stability and keep the viscosity manageable for injection in a convenient volume, e.g., in a high-concentration formulation suitable for subcutaneous administration.

According to one embodiment, the stable pharmaceutical formulation comprises: (i) a human IgG4 antibody that specifically binds to human LAG-3, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of up to 250 mg/ml±45 mg/mL; (ii) histidine buffer at 10 mM±2 mM, which buffers at pH 6.0±0.3; (iii) polysorbate 80 at 0.1%±0.05% w/v to 0.2% w/v±0.1% w/v; (iv) sucrose at 5%±1% w/v to 10%±2% w/v; and (v) from 1 mM±0.2 mM to 100 mM±20 mM arginine hydrochloride.

According to one embodiment, the stable pharmaceutical formulation comprises: (i) a human IgG4 antibody that specifically binds to human LAG-3, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 175 mg/ml±26.25 mg/mL; (ii) histidine buffer at 10 mM±2 mM, which buffers at pH 6.0±0.3; (iii) polysorbate 80 at 0.1% w/v±0.05% w/v; (iv) sucrose at 10%±2% w/v; and (v) from 1 mM±0.2 mM to 100 mM±20 mM arginine hydrochloride.

According to one embodiment, the stable pharmaceutical formulation comprises: (i) a human IgG4 antibody that specifically binds to human LAG-3, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 150 mg/ml±22.5 mg/mL; (ii) histidine buffer at 10 mM±2 mM, which buffers at pH 6.0±0.3; (iii) polysorbate 80 at 0.1% w/v±0.05% w/v; (iv) sucrose at 10%±2% w/v; and (v) from 1 mM±0.2 mM to 100 mM±20 mM arginine hydrochloride.

According to one embodiment, the stable pharmaceutical formulation comprises: (i) a human IgG4 antibody that specifically binds to human LAG-3, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 100 mg/mL±15 mg/mL; (ii) histidine buffer at 10 mM±2 mM, which buffers at pH 6.0±0.3; (iii) sucrose at 10% w/v±2% w/v; (iv) polysorbate 80 at 0.1% w/v±0.05%; and (v) from 1 mM±0.2 mM to 100 mM±20 mM arginine hydrochloride.

According to one embodiment, the stable pharmaceutical formulation comprises: (i) a human IgG4 antibody that specifically binds to human LAG-3, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 50 mg/mL±7.5 mg/mL; (ii) histidine buffer at 10 mM±2 mM, which buffers at pH 6.0±0.3; (iii) sucrose at 10% w/v±2% w/v; (iv) polysorbate 80 at 0.1% w/v±0.05%; and (v) 20 mM±4 mM arginine hydrochloride.

According to one embodiment, the stable pharmaceutical formulation comprises: (i) a human IgG4 antibody that specifically binds to human LAG-3, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 25 mg/mL±3.75 mg/mL; (ii) histidine buffer at 10 mM±2 mM, which buffers at pH 6.0±0.3; (iii) sucrose at 10% w/v±2% w/v; (iv) polysorbate 80 at 0.1% w/v±0.05%; and (v) 20 mM±4 mM arginine hydrochloride.

Additional non-limiting examples of pharmaceutical formulations encompassed by the present invention are set forth elsewhere herein, including the working Examples presented below.

### Containers and Methods of Administration

The pharmaceutical formulations of the present disclosure may be contained within any container suitable for storage of medicines and other therapeutic compositions. For example, the pharmaceutical formulations may be contained within a sealed and sterilized plastic or glass container having a defined volume such as a vial, ampule, syringe, cartridge, or bottle. Different types of vials can be used to contain the formulations of the present disclosure including, e.g., clear and opaque (e.g., amber) glass or plastic vials. Likewise, any type of syringe can be used to contain or administer the pharmaceutical formulations of the present disclosure.

The pharmaceutical formulations of the present disclosure may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than or equal to 500 parts per billion (ppb) of tungsten. The term “low tungsten” means that the syringe contains less than 500 ppb of tungsten. For example, a low tungsten syringe, according to the present disclosure, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial, or to preserve their stability. Thus, pharmaceutical formulations of the present disclosure, according to certain embodiments, may be contained within a syringe that comprises a coated plunger, or within a vial that is sealed with a coated rubber stopper. For example, the plunger or stopper may be coated with a fluorocarbon film.

Examples of coated stoppers or plungers suitable for use with vials and syringes containing the pharmaceutical formulations of the present disclosure are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present disclosure are commercially available under the tradename “FluroTec®”, available from West Pharmaceutical Services, Inc. (Lionville, PA). FluroTec® is an example of a fluorocarbon coating used to minimize or prevent drug product from adhering to the rubber surfaces.

According to certain embodiments of the present disclosure, the pharmaceutical formulations may be contained within a low tungsten syringe that comprises a fluorocarbon-coated plunger.

The pharmaceutical formulations can be administered to a patient by parenteral routes such as injection (e.g., subcutaneous, intravenous, intramuscular, intraperitoneal, etc.) or percutaneous, mucosal, nasal, pulmonary or oral administration. Numerous reusable pen or autoinjector delivery devices can be used to subcutaneously deliver the pharmaceutical formulations of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ 1, ∥ and Ill (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany). Examples of disposable pen or autoinjector delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park, IL).

The use of a microinfusor to deliver the pharmaceutical formulations of the present disclosure is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) or viscous solutions.

In certain embodiments, the stable liquid pharmaceutical formulation of any of the preceding aspects is contained in a sterile glass vial and is administered as an IV infusion.

In one embodiment, the container is a 20 mL type 1 clear borosilicate glass vial. In certain embodiments, the container is a 2 mL, 5 mL or 10 mL type 1 borosilicate glass vial with a chlorobutyl stopper, with a FluroTec® coating.

In one embodiment, the liquid pharmaceutical formulation of the present disclosure comprising about 25 mg/mL or 50 mg/mL of mAb1 is administered intravenously and may be contained in a glass vial. In some aspects, the glass vial contains 400 mg mAb1. In some aspects, the glass vial contains 800 mg mAb1.

In one embodiment, the liquid pharmaceutical formulation of the present disclosure comprising about 100 mg/mL of mAb1 is administered subcutaneously and may be contained in a glass vial.

In certain embodiments, the present disclosure provides an autoinjector comprising any of the liquid formulations described herein. In some embodiments, the present disclosure provides an autoinjector comprising a stable liquid formulation comprising about 50 mg/mL, about 100 mg/mL, about 150 mg/mL or about 175 mg/mL of mAb1, about 10 mM of histidine, at pH of about 6.0, about 5% sucrose or about 10% sucrose, about 20 mM arginine hydrochloride, and about 0.1% polysorbate 80 or about 0.2% polysorbate 80. In some aspects, the autoinjector contains 400 mg mAb1. In some aspects, the autoinjector contains 800 mg mAb1. In some aspects, the autoinjector contains 1600 mg mAb1.

In certain embodiments, the present disclosure provides a prefilled syringe comprising any of the liquid formulations described herein. In some embodiments, the present disclosure provides a prefilled syringe comprising a stable liquid formulation comprising about 50 mg/mL, about 100 mg/mL, about 150 mg/mL or about 175 mg/mL of mAb1, about 10 mM of histidine, at pH of about 6.0, about 5% sucrose or about 10% sucrose, about 20 mM arginine hydrochloride, and about 0.1% polysorbate 80 or about 0.2% polysorbate 80. In certain embodiments, the syringe is a 1 mL or 2.25 mL long glass syringe filled with a 27-gauge thin wall needle, a fluorocarbon coated rubber plunger and a rubber needle shield. In some aspects, the prefilled syringe contains 400 mg mAb1. In some aspects, the prefilled syringe contains 800 mg mAb1. In some aspects, the autoinjector contains 1600 mg mAb1.

In one embodiment, the liquid pharmaceutical formulation containing about 50 mg/mL±7.5 mg/mL mAb1 is administered in a volume of approximately up to 2 mL in a prefilled syringe. In certain embodiments, the syringe is a 1 mL or 2.25 mL long glass syringe filled with a 27-gauge thin wall needle, a fluorocarbon coated rubber plunger and a rubber needle shield. In one embodiment, the syringe is an OMPI 1 mL long glass syringe fitted with a 27-gauge needle, a FM27 rubber needle shield, and a FLUROTEC® coated 4023/50 rubber plunger.

In one embodiment, the liquid pharmaceutical formulation containing about 100 mg/mL±15 mg/mL anti-LAG-3 antibody is administered in a volume of approximately up to 2 mL in a prefilled syringe. In one embodiment, the syringe is a 1 mL or 2.25 mL long glass syringe filled with a 27-gauge thin wall needle, a fluorocarbon coated rubber plunger and a rubber needle shield. In one embodiment, the syringe is an OMPI 1 mL long glass syringe fitted with a 27-gauge needle, a FM27 rubber needle shield, and a FLUROTEC® coated 4023/50 rubber plunger.

### Therapeutic Uses of the Pharmaceutical Formulations

The pharmaceutical formulations of the present disclosure are useful, inter alia, for the treatment, prevention or amelioration of any disease or disorder associated with LAG-3 activity, including diseases or disorders mediated by LAG-3. Exemplary, non-limiting diseases and disorders that can be treated or prevented by the administration of the pharmaceutical formulations of the present disclosure include viral infections, autoimmune diseases and various cancers such as, e.g., brain cancer, lung cancer, prostate cancer, colorectal cancer, head and neck cancer, skin cancer, various blood cancers, and endometrial cancers.

### EXAMPLES

The following examples are presented so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by mole, molecular weight is average molecular weight, temperature is in degrees Centigrade, and pressure is at or near atmospheric pressure.

### Example 1: Development of an Anti-LAG-3 Antibody Formulation

The goals of the formulation activities were to develop a formulation with the following attributes:
A liquid formulation with a concentration of the anti-LAG-3 antibody sufficient to deliver a dose of 50 mg, 400 mg, 800 mg, 1600 mg or more;
A near iso-osmolar formulation that is stable upon dilution with commonly used diluents, e.g., 0.9% sodium chloride injection or 5% dextrose injection, for intravenous infusion;
A formulation that is compatible with and stable in Type 1 clear glass vial and standard serum stopper as packaging; and
A sterile drug product (DP) solution that supports long-term stability;
   A formulation that minimizes antibody high molecular weight (HMW) species when subjected to handling and thermal stresses;
   A formulation that minimizes changes in the relative distribution of antibody charged species when subjected to thermal stress; and
   A formulation that maintains biological activity when subjected to handling and thermal stress.

Throughout formulation development, three primary protein stress conditions (representing extreme handling conditions beyond which the antibody drug product would not be subjected during handling, manufacturing, shipping, storing, and labeling) were employed to develop and optimize the antibody formulations and to evaluate the effects of potential real-world stresses on the stability of the drug product. These stress conditions included:
Agitation (vortexing) of the protein solution at room temperature. Vortexing in glass vials exceeds the agitation that occurs during the handling and manufacturing of the protein.
Incubating the protein solution at elevated temperature (37° C., 40° C. or 45° C.) relative to the proposed DP storage condition (2° C.-8° C.).
Subjecting the protein to multiple freeze thaw cycles. Since the protein will undergo at least one freeze thaw cycle during the manufacture of DP, multiple freeze thaw cycles simulate and exceed the actual stress the protein is expected to experience.

Other attributes of the formulations will be apparent from the description herein.

### Anti-LAG-3 Antibodies:

Anti-LAG-3 antibodies are described in U.S. Pat. No. 10,358,495, incorporated herein in its entirety. The exemplary antibody used in the Examples below is a fully human anti-LAG-3 antibody known as “REGN3767” or “Fianlimab” comprising the heavy chain complementarity determining regions (HCDRs) within the heavy chain variable region (HCVR) amino acid sequence of SEQ ID NO: 1 and the light chain complementarity determining regions (LCDRs) within the light chain variable region (LCVR) amino acid sequence of SEQ ID NO: 2; or an HCVR/LCVR amino acid sequence pair comprising SEQ ID NOs: 1/2; or heavy and light chain CDR sequences comprising SEQ ID NOs: 3-8; or the heavy chain CDRs within a heavy chain amino acid sequence of SEQ ID NO: 9 and the light chain CDRs within a light chain amino acid sequence of SEQ ID NO: 10; or a heavy chain amino acid sequence of SEQ ID NO: 9 and a light chain amino acid sequence of SEQ ID NO: 10; and herein referred to as “mAb1”. The antibody possesses an approximate molecular weight of about 145 kDa and the heavy chain possesses an IgG4 isotype constant region.

mAb1 was evaluated for intravenous (IV) and subcutaneous (SC) administration in a first-in-human study. Initially, a single, dual-use lyophilized formulation was developed such that lyophilized mAb1 drug product (DP) can be reconstituted with sterile water for injection to a concentration of either 50 mg/mL mAb1 for IV infusion or 100 mg/mL mAb1 for SC injection.

Formulation development activities involved assessment of buffers, pH, organic co-solvents, surfactants, and sucrose (as the thermal stabilizer) to identify excipients that enhance protein stability. In the early phase of formulation development, screening for buffers and pH was carried out. Three buffers were tested: acetate (pH 4.5-5.5), histidine (pH 5.5-6.50 and phosphate (pH 6.0-7.0). 10 mM histidine at pH 6.0 showed optimum stability under accelerated conditions and was chosen going forward. Three surfactants/cosolvents were tested: polysorbate 20, polysorbate 80 and PEG3350. PS80 was selected for the formulation. Results generated from these studies were used to develop a stable lyophilized formulation, containing 50 mg/mL mAb1, 10 mM histidine, pH 6.0, 5% (w/v) sucrose, and 0.1% (w/v) polysorbate 80, that was suitable for reconstitution to the liquid form prior to clinical use.

For later stage clinical development, formulation development studies were conducted to develop a liquid DP formulation of mAb1. The information gained during the early phase formulation development was the basis for determining buffer and pH in the late phase formulation.

### Buffer and pH

The effect of buffer and pH on the thermal stability was examined in liquid formulations by incubating a 25 mg/mL mAb1 formulation at 45° C. for 28 days in a series of buffer systems at varying pH ranges (Table 1). L-histidine buffer at 10 mM and pH of 6.0 was selected as the formulation buffer because it provided the best overall level of protein stabilization with respect to formation of low molecular weight (LMW) and high molecular weight (HMW) species and the formation of charge variants.

TABLE 1
Effect of pH on the Stability of 25 mg/mL mAb1 Incubated at 45° C. for 14 Days

| | |
|---|---|
| Formulation | 25 mg/mL mAb1, 10 mM buffer |
| Fill Volume | 0.4 mL |
| Container/Closure | 2 mL Type 1 glass vials with 13 mm FluroTec ®-coated elastomeric stopper |

| | | Turbidity | % Protein | Change in Purity by | | | Change in Charge Variants | | |
|---|---|---|---|---|---|---|---|---|---|
| | | (Increase | Recovered | SE-UPLC^{b} | | | by CEX-UPLC^{b} | | |
| 10 mM Buffer | Color and | in OD at | by | % | % | % | % | % | % |
| and pH | Appearance^{a} | 405 nm) | RP-UPLC | HMW | Main | LMW | Acidic | Main | Basic |
| Phosphate pH 6.5 | Pass | 0.12 | 91 | 2.1 | −2.3 | 0.1 | 12.8 | −9.4 | −3.3 |
| Phosphate pH 6.0 | Pass | 0.00 | 95 | 2.0 | −2.1 | 0.1 | 7.9 | −5.1 | −2.8 |
| Histidine pH 6.5 | Pass | 0.01 | 99 | 1.6 | −1.7 | 0.1 | 8.0 | −6.5 | −1.5 |
| Histidine pH 6.0 | Pass | 0.00 | 102 | 1.2 | −1.2 | 0.1 | 4.5 | −4.1 | −0.4 |
| Histidine pH 5.5 | Pass | 0.00 | 96 | 1.2 | −1.3 | 0.1 | 8.4 | −10.0 | 1.6 |
| Acetate pH 5.5 | Pass | 0.00 | 95 | 1.3 | −1.3 | 0.1 | 6.8 | −6.0 | −0.8 |
| Acetate pH 5.0 | Pass | 0.00 | 96 | 1.3 | −1.4 | 0.2 | 13.7 | −13.3 | −0.5 |

^{a}Pass if clear to slightly opalescent, essentially free from visible particulates, and colorless to pale yellow
^{b}Reported as a change in purity relative to the starting material. The starting material (no incubation) contains ≥98.5% native peak by SE-UPLC and ≥57.2% main peak by CEX-UPLC in all formulations.
CEX, cation exchange;
DS, drug substance;
HMW, high molecular weight;
LMW, low molecular weight;
OD, optical density;
Ph. Eur., European Pharmacopoeia;
RP, reversed-phase;
SE, size-exclusion;
UPLC, ultra-performance liquid chromatography;
USP, United States Pharmacopeia

### Surfactant

Selected surfactants/co-solvent (polysorbate 20, polysorbate 80, and PEG3350) were elevated for effect on agitation stress of 25 mg/mL mAb1. All of the surfactants/co-solvents adequately protected protein from agitation-induced instability, with no observed increases in HMW species. Polysorbate 80 was chosen as the surfactant for the DP formulation because it stabilized the protein to agitation stress and has a demonstrated safety profile for use in monoclonal antibody formulations (Table 2).

TABLE 2
Effect of Surfactants on the Stability of 25 mg/mL mAb1 after Vortexing (120 minutes at 1000 RPM)

| Formulation | | 25 mg/mL mAb1, 10 mM histidine, 5% (w/v) sucrose, pH 6.0 | | | | | | | | |
|---|---|---|---|---|---|---|---|---|---|---|
| Fill Volume | | 0.4 mL | | | | | | | | |
| Container/Closure | | 2 mL Type 1 glass vials (2R glass vials) with 13 mm FluroTec ®-coated elastomeric stopper | | | | | | | | |
| | | Turbidity | | % Protein | Change in Purity | | | Change in Charge Variants | | |
| Surfactant/ | Color and | (Increase in OD | | Recovered by | by SE-UPLC^{b} | | | by CEX-UPLC^{b} | | |
| Cosolvent | Appearance^{a} | at 405 nm) | pH | RP-UPLC | % HMW | % Main | % LMW | % Acidic | % Main | % Basic |
| No surfactant/ | Pass | 0.00 | 6.0 | 101 | 0.7 | −0.5 | −0.2 | 0.6 | 0.0 | −0.6 |
| Cosolvent | | | | | | | | | | |
| 0.1% (w/v) | Pass | 0.00 | 6.0 | 92 | 0.0 | 0.1 | −0.1 | 0.5 | −0.2 | −0.4 |
| polysorbate 20 | | | | | | | | | | |
| 0.1% (w/v) | Pass | 0.00 | 6.0 | 94 | 0.0 | 0.0 | −0.1 | 0.4 | −0.2 | −0.2 |
| polysorbate 80 | | | | | | | | | | |
| 1% PEG3350 | Pass | 0.00 | 6.0 | 99 | 0.0 | 0.0 | 0.0 | 0.6 | −0.4 | −0.2 |

^{a}Pass if clear to slightly opalescent, essentially free from visible particulates, and colorless to pale yellow.
^{b}Reported as a change in purity relative to the starting material. The starting material (no incubation) contains ≥97.7% main peak by SE-UPLC and ≥60.5% main peak by CEX UPLC in all formulations.
CEX, cation exchange;
DS, drug substance;
HMW, high molecular weight;
LMW, low molecular weight;
OD, optical density;
Ph. Eur., European Pharmacopoeia;
RP, reversed-phase;
SE, size-exclusion;
UPLC, ultra-performance liquid chromatography;
USP, United States Pharmacopeia

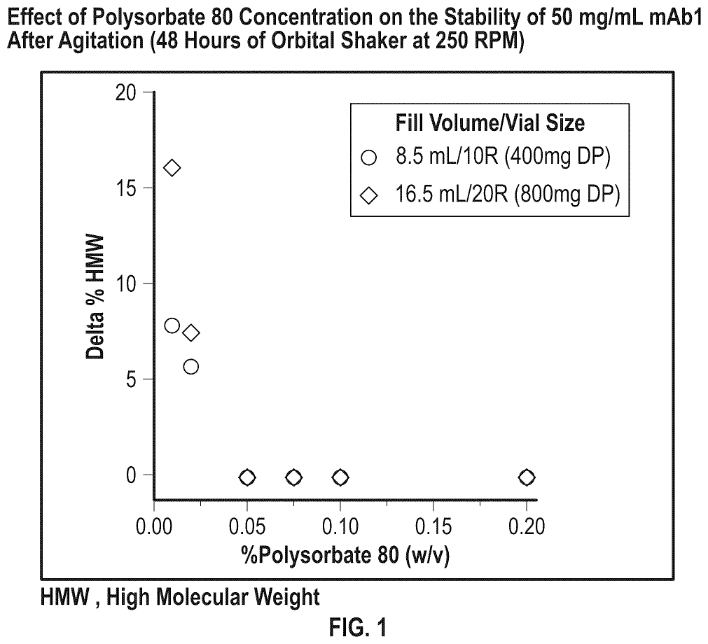

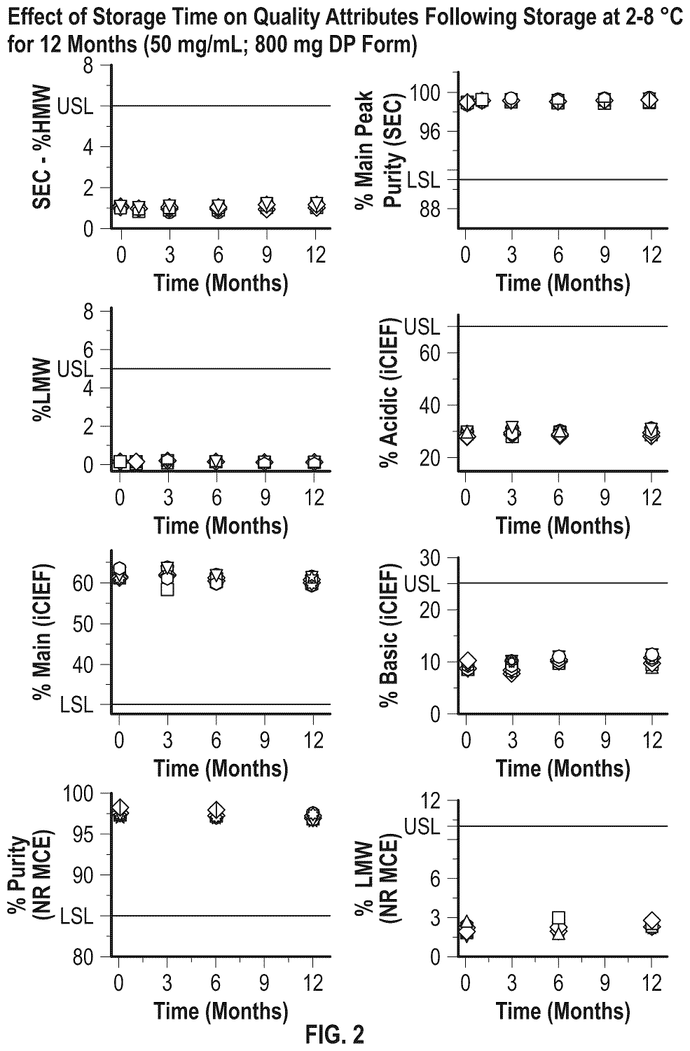

To optimize the polysorbate 80 concentration, an agitation study using a range of polysorbate 80 concentrations (0.01% to 0.20% w/v) was performed. The results indicated that mAb1 with 0.10% (w/v) polysorbate 80 falls in a stable region of the characterized range with sufficient tolerance on either side to tolerate variations in the polysorbate 80 concentration (FIG. 1). The polysorbate 80 concentration of 0.10% (w/v) is consistent with the final polysorbate 80 concentration in the reconstituted IV formulation for the initial clinical trial studies.

### Thermal Stabilizer

Stabilizers such as sucrose are often added to antibody formulations to increase the thermal stability of proteins. The influence of sucrose was studied by subjecting a 25 mg/mL formulation with or without 5% (w/v) sucrose to accelerated thermal stress conditions (Table 3). After incubation at 45° C. for 28 days, HMW species increased by 2.0% and 1.5%, in control and sucrose formulation, respectively.

TABLE 3
Effect of Sucrose on the Stability of 25 mg/mL mAb1 Incubated at 45° C. for 28 Days

| | |
|---|---|
| Formulation | 25 mg/mL mAb1, 10 mM histidine, pH 6.0 |
| Fill Volume | 0.4 mL |
| Container/Closure | 2 mL Type 1 glass vials with 13 mm FluroTec ®-coated elastomeric stopper |

| | | Turbidity | | % Protein | Change in Purity | | | Change in Charge Variants | | |
|---|---|---|---|---|---|---|---|---|---|---|
| | | (Increase | | Recovered | by SE-UPLC^{b} | | | by CEX-UPLC^{b} | | |
| | Color and | in OD | | by RP- | % | % | % | % | % | % |
| Stabilizer | Appearance^{a} | at 405 nm) | pH | UPLC | HMW | Main | LMW | Acidic | Main | Basic |
| No sucrose | Pass | 0.01 | 6.1 | 94 | 2.0 | −2.0 | 0.1 | 15.3 | −15.7 | 0.4 |
| 5% (w/v) | Pass | 0.00 | 6.1 | 95 | 1.5 | −1.5 | 0.1 | 15.5 | −16.0 | 0.5 |
| sucrose | | | | | | | | | | |

^{a}Pass if clear to slightly opalescent, essentially free from visible particulates, and colorless to pale yellow.
^{b}Reported as a change in purity relative to the starting material. The starting material (no incubation) contains ≥97.7% main peak by SE-UPLC and ≥60.5% main peak by CEX UPLC in both formulations.
CEX, cation exchange;
DS, drug substance;
HMW, high molecular weight;
LMW, low molecular weight;
OD, optical density;
Ph. Eur., European Pharmacopoeia;
RP, reversed-phase;
SE, size-exclusion;
UPLC, ultra-performance liquid chromatography;
USP, United States Pharmacopeia

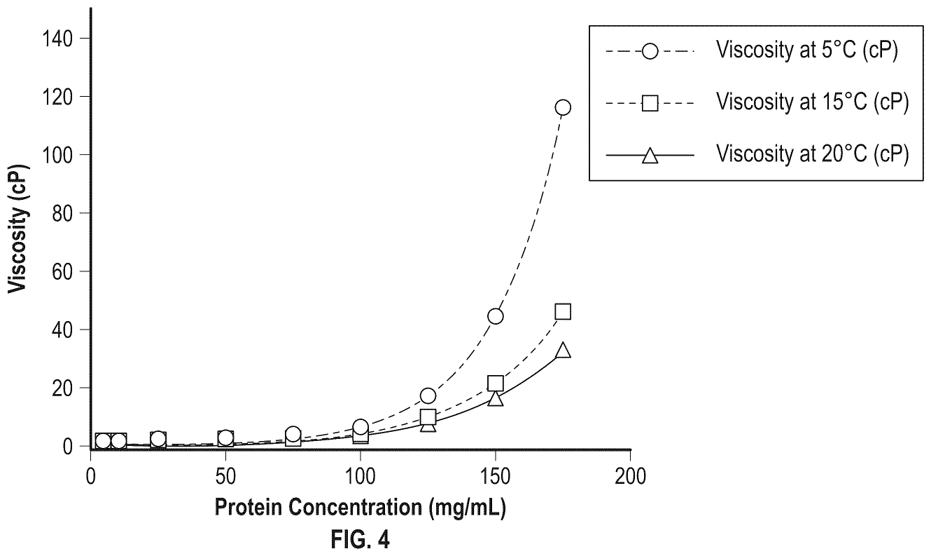

An assessment of different stabilizers and concentrations, using mAb1 drug substance (DS), to develop a liquid formulation at 50 mg/mL mAb1 was conducted. As a part of this study, the following stabilizers and combinations were evaluated: 5% (w/v) sucrose, 5% (w/v) sucrose with 20 mM arginine hydrochloride, and 10% (w/v) sucrose with 20 mM arginine hydrochloride. A summary of stability results is presented in Table 4. Based on the SE-UPLC and CEX-UPLC analysis, all three formulations imparted similar levels of protein stabilization for mAb1. Among the three formulations, 10% (w/v) sucrose with 20 mM arginine hydrochloride yielded the best overall level of protein stabilization with respect to formation of low molecular weight (LMW) and high molecular weight (HMW) species and formation of charge variants. The addition of L-arginine hydrochloride showed no negative impact to stability. L-arginine hydrochloride was included in the 50 mg/mL formulation since a high concentration drug substance (DS) was targeted to support the development of both an IV formulation and a subcutaneous (SC) formulation at a higher mAb1 concentration. In order to manufacture ≥175 mg/mL mAb1 DS, L-arginine hydrochloride was added as a viscosity reducer. Table 5 describes the effect of stabilizers of a high-concentration formulation, and FIG. 4 illustrates change in viscosity at various antibody concentrations and storage temperatures. An anti-LAG3 antibody formulation containing 175 mg/mL antibody in 10 mM histidine, 5% sucrose, 70 mM arginine HCl, 0.1% PS80, pH 6 and held at 20° C., viscosity was no greater than 25 cP.

The formulation containing 50 mg/mL mAb1, 10 mM histidine, 10% (w/v) sucrose, 20 mM arginine hydrochloride, and 0.1% polysorbate 80 at pH 6.0 was selected as mAb1 DP formulation.

TABLE 4
Effect of Stabilizers on the Stability of 50 mg/mL mAb1 Incubated at 40° C. for 28 Days

| | |
|---|---|
| Formulation | 50 mg/mL mAb1, 10 mM histidine, pH 6.0, 0.1% polysorbate 80 |
| Fill Volume | 1 mL |
| Container/Closure | 2 mL Type 1 borosilicate glass vial with a FluroTec ®-coated 4432/50 butyl rubber stopper |

| | | | % Total | | | | Charge Variants | | |
|---|---|---|---|---|---|---|---|---|---|
| | | Turbidity | Protein | Purity by SE-UPLC^{a} | | | by CEX-UPLC^{a} | | |
| | | (Increase | Recovered | | % Main | | % | % | % |
| | Color and | in OD at | by | % HMW | Peak | % LMW | Region | Region | Region |
| pH/Buffer | Appearance | 405 nm) | RP-UPLC | Species | Purity | Species | 1 | 2 | 3 |
| 5% | Pass | 0.00 | 98 | 0.8 | −0.9 | 0.2 | 10.5 | −10.2 | −0.3 |
| Sucrose | | | | | | | | | |
| 5% | Pass | 0.00 | 98 | 0.8 | −1.0 | 0.2 | 10.6 | −10.6 | −0.1 |
| Sucrose, | | | | | | | | | |
| 20 mM | | | | | | | | | |
| Arginine | | | | | | | | | |
| HCl | | | | | | | | | |
| 10% | Pass | 0.00 | 100 | 0.6 | −0.8 | 0.2 | 10.5 | −10.7 | −0.2 |
| Sucrose, | | | | | | | | | |
| 20 mM | | | | | | | | | |
| Arginine | | | | | | | | | |
| HCl | | | | | | | | | |

^{a}Reported as a change in purity relative to the starting material. The starting material (no incubation) contains ≥98.5% native peak by SE-UPLC and ≥57.3% main peak by CEX-UPLC in all formulations.
CEX, cation exchange;
DS, drug substance;
HMW, high molecular weight;
LMW, low molecular weight;
OD, optical density;
RP, reversed-phase;
SE, size-exclusion;
UPLC, ultra performance liquid chromatography

TABLE 5
Effect of Stabilizers on the Stability of 175 mg/mL REGN3767 Incubated at 2-8° C. for 36 Months

| | |
|---|---|
| Formulation | 175 mg/mL REGN3767, 10 mM histidine, 0.1% polysorbate 80, pH 6.0 |
| Fill Volume | 1 mL |
| Container/Closure | 2 mL Type 1 borosilicate glass vial with a FluroTec ®-coated 4432/50 butyl rubber stopper |

| | | | | | Change in Purity | | | | | |
|---|---|---|---|---|---|---|---|---|---|---|
| | | | | | by SE-UPLC^{a} | | | Change in Charge Variants | | |
| | | | | Turbidity | | % | | by CEX-UPLC | | |
| | Viscosity | | | (Increase | | Main | | % | % | % |
| Sucrose/ | at 20° C. | Color and | | in OD at | % | Peak | % | Region | Region | Region |
| Arginine HCl | (cP) | Appearance | pH | 405 nm) | HMW | Purity | LMW | 1 | 2 | 3 |
| 5% Sucrose, | 31.1 | Pass^{b} | 6.1 | 0.02 | 0.6 | −0.7 | 0.1 | 2.2 | −1.2 | −1.1 |
| 50 mM Arginine HCl | | | | | | | | | | |
| 0% Sucrose, | 19.4 | Pass | 6.1 | 0.00 | 0.6 | −0.7 | 0.0 | 2.2 | −1.5 | −0.9 |
| 150 mM Arginine HCl | | | | | | | | | | |
| 2% Sucrose, | 25.2 | Pass | 6.1 | 0.00 | 0.6 | −0.7 | 0.0 | 2.1 | −1.6 | −0.4 |
| 100 mM Arginine HCl | | | | | | | | | | |
| 5% Sucrose, | 25.0 | Pass | 6.1 | 0.02 | 0.5 | −0.6 | 0.1 | 0.3 | 0.3 | −0.7 |
| 100 mM Arginine HCl | | | | | | | | | | |

^{a}Reported as a change in purity relative to the starting material. The starting material (no incubation) contains ≥98.0% native peak by SE-UPLC and ≥57.3% main peak by CEX-UPLC in all formulations.
^{b}Pass if clear to slightly opalescent, essentially free from visible particulates, and colorless to pale yellow.
CEX, cation exchange;
DS, drug substance;
HMW, high molecular weight;
LMW, low molecular weight;
OD, optical density;
SE, size-exclusion;
UPLC, ultra performance liquid chromatography

### Example 2: Exemplary Formulations

In certain embodiments, mAb1 is formulated as an aqueous buffered formulation containing from 5 mg/ml±0.75 mg/ml to 250 mg/ml±45.0 mg/ml mAb1, 10 mM±2 mM histidine buffer, 0.05%±0.05% to 0.2%±0.1% w/v polysorbate, 1%±0.2% to 15%±3% w/v sucrose, and from 1 mM±0.2 mM to 100 mM±20 mM arginine hydrochloride, at pH 6.0±0.3.

Exemplary formulations include:
A stable pharmaceutical formulation comprising: 25 mg/ml±3.75 mg/mL mAb1, 10±2 mM histidine buffer, 0.1%±0.05% w/v polysorbate 80, 5%±1% or 10%±2% w/v sucrose, and 20 mM±4 mM arginine hydrochloride at pH 6.0±0.3.
A stable pharmaceutical formulation comprising: 25 mg/ml±3.75 mg/mL mAb1, 10±2 mM histidine buffer, 0.1%±0.05% w/v polysorbate 80, 5%±1% w/v sucrose at pH 6.0±0.3.
A stable pharmaceutical formulation comprising: 50 mg/ml±7.5 mg/mL mAb1, 10±2 mM histidine buffer, 0.1%±0.05% w/v polysorbate 80, 5%±1% or 10%±2% w/v sucrose, and 20 mM±4 mM arginine hydrochloride at pH 6.0±0.3.
A stable pharmaceutical formulation comprising: 50 mg/ml±7.5 mg/mL mAb1, 10±2 mM histidine buffer, 0.1%±0.05% w/v polysorbate 80, 5%±1% w/v sucrose at pH 6.0±0.3.
A stable pharmaceutical formulation comprising: 100±15 mg/mL mAb1, 10±2 mM histidine buffer, 0.1%±0.05% w/v polysorbate 80, 10%±2% w/v sucrose, and 10 mM±2 mM to 100 mM±20 mM arginine hydrochloride, at pH 6.0±0.3.
A stable pharmaceutical formulation comprising: 150±22.5 mg/mL mAb1, 10±2 mM histidine buffer, 0.2%±0.1% w/v polysorbate 80, 10%±2% w/v sucrose, and 10 mM±2 mM to 100 mM±20 mM arginine hydrochloride, at pH 6.0±0.3.
A stable pharmaceutical formulation comprising: 175±37.5 mg/mL mAb1, 10±2 mM histidine buffer, 0.1%±0.05% w/v polysorbate 80, 10%±2% w/v sucrose, and 10 mM±2 mM to 100 mM±20 mM arginine hydrochloride, at pH 6.0±0.3.

### Example 3: Methods Used to Assess Formulation Stability

The following assays were applied to assess formulation stability:
Color and appearance by visual inspection
pH
Turbidity measured by increase in OD at 405 nm Subvisible particulate analysis by Micro-Flow Imaging™ (MFI) and light obscuration by HIAC
Protein concentration by reversed-phase ultra performance liquid chromatography (RP-UPLC)
Purity of DP was assessed using the following assays:
   Size-exclusion ultra performance liquid chromatography (SE-UPLC)
   Reduced and non-reduced microchip capillary electrophoresis-sodium dodecyl sulfate (MCE-SDS)
Charge variant analysis was determined using the following assays:
   Cation exchange UPLC (CEX-UPLC)
   Imaged capillary isoelectric focusing (iCIEF)
Charge variants are reported as percent Region 1, Region 2, and Region 3. Region 1 corresponds to acidic species which elute before the main peak; Region 2 corresponds to the main peak; and Region 3 corresponds to basic species which elute after the main peak.
Potency was assessed by bioassay: the relative potency of each sample was determined by bioassay and is defined as: (IC₅₀ reference sample/IC₅₀ sample)×100%. The measured potency of storage stability samples must be within 50%-150% of the measured potency of the reference standard.

The physical stability of a formulation refers to properties such as color, appearance, pH, turbidity, and protein concentration. The presence of visible particulates in solution can be detected by visual inspection. A solution passes visual inspection if it is clear to slightly opalescent, essentially free from visible particulates, and colorless to pale yellow. In addition, turbidity, measured by OD at 405 nm, can also be used to detect particulates in solution. An increase in OD at 405 nm may indicate the presence of particulates, an increase in opalescence, or color change of the test articles. MFI is used to measure subvisible particulates that are ≥2 μm in size. The protein concentration of mAb1 is measured by a RP-UPLC assay and reported as percent protein recovery relative to the starting material. In the RP-UPLC assay, mAb1 is eluted from the RP column as a single peak. The protein concentration is determined from the mAb1 total peak area by comparing it with a calibration curve generated using mAb1 standards. Percent of recovery is calculated based on the measured protein concentration relative to the starting protein concentration.

Chemical stability refers to the formation of covalently modified forms (e.g. covalent aggregates, cleavage products, or charge variant forms) and non-covalently modified forms (e.g. non-covalent aggregates) of protein. Higher and lower molecular weight degradation products can be separated from native mAb1 by SE-UPLC and MCE-SDS methods. The percentage of degraded mAb1 in the SE-UPLC and MCE-SDS methods is calculated from the ratio of the area of all non-native peaks to the total area of all mAb1 peaks. Charge variant forms of mAb1 are resolved using CEX-UPLC and iCIEF.

In the CEX-UPLC method, peaks with retention times earlier than that of the main peak are labeled as “acidic” peaks; the peaks with retention times later than that of the main peak are labeled as “basic” peaks. In the iCIEF method, peaks that are focused to a pI lower than that of the main peak are labeled “acidic” peaks, whereas those focused to a pI higher than that of the main peak are labeled “basic” peaks.

### Example 4: Stability of the 50 mg/ml Formulation when Stored Inverted

The formulation containing 50 mg/mL mAb1, 10 mM L-histidine, 10% (w/v) sucrose, 20 mM L-arginine hydrochloride, and 0.1% polysorbate 80 at pH 6.0 was selected as the liquid mAb1 formulation for co-administration with cemiplimab and for manufacturing mAb1. The suitability of this formulation was demonstrated by no appreciable change in the physical or chemical stability for any of the monitored attributes after 42 months storage at 5° C. (2-8° C.) as shown in Table 6.

TABLE 6
Stability of mAb1 Drug Product 50 mg/mL, Stored at 2-8° C., Inverted
Sterile, vialed mAb1 recombinant protein, 50.0 mg/mL, in an aqueous buffered
solution, pH 6.0, containing 20 mM arginine hydrochloride, 10 mM histidine,
10% (w/v) sucrose, and 0.1% polysorbate 80
USP/Ph. Eur. Type I clear glass 20R vial with a 20 mm FluroTec ®-
coated 4432/50 elastomer stopper and 20 mm Flip-Off ® seal
5° C. Inverted

| Assay | t = 0^{a} | 1 | 3 | 6 | 9 | 12 | 18 | 24 | 30 | 36 | 42 |
|---|---|---|---|---|---|---|---|---|---|---|---|
| Physical form/Condition | LEFVP | LEFVP | LEFVP | LEFVP | LEFVP | LEFVP | LEFVP | LEFVP | LEFVP | LEFVP | LEFVP |
| Clarity | Not > | Not > | Not > | Not > | Not > | Not > | Not > | Not > | Not > | Not > | Not > |
| | III | III | II | I | III | II | II | III | III | II | III |
| Color | Not > | Not > | Not > | Not > | Not > | Not > | Not > | Not > | Not > | Not > | Not > |
| | BY7 | BY6 | BY7 | BY6 | BY5 | BY7 | BY7 | BY6 | BY7 | BY7 | BY7 |
| pH | 6.0 | 6.0 | 6.1 | 6.1 | 6.0 | 6.0 | 6.0 | 6.0 | 6.0 | 6.0 | 6.0 |
| Total Protein (A₂₈₀) | 51.3 | 50.7 | 50.4 | 51.3 | 51.4 | 50.6 | 50.9 | 50.6 | 54.2b | 50.8 | 51.1 |
| Potency (%) | 96 | 98 | 132 | 107 | 98 | 115 | 117 | 87 | 107 | 113 | 102 |
| Purity by Reduced MCE(^{%)} | a. 97 | a. 97 | a. 97 | a. 97 | a. 97 | a. 98 | a. 98 | a. 98 | a. 96 | a. 97 | a. 96 |
| | b. 0 | b. 0 | b. 0 | b. 0 | b. 0 | b. 0 | b. 0 | b. 0 | b. 0 | b. 0 | b. 0 |
| | c. 2 | c. 2 | c. 2 | c. 2 | c. 3 | c. 2 | c. 2 | c. 2 | c. 4 | c. 3 | c. 3 |
| Purity by Non-Reduced MCE | a. 98 | a. 98 | a. 97 | a. 98 | a. 97 | a. 98 | a. 98 | a. 97 | a. 96 | a. 97 | a. 96 |
| (%) | b. 2 | b. 2 | b. 2 | b. 2 | b. 3 | b. 2 | b. 2 | b. 3 | b. 2 | b. 1 | b. 2 |
| | c. 0 | c. 0 | c. 0 | c. 0 | c. 0 | c. 0 | c. 0 | c. 0 | c. 1 | c. 1 | c. 1 |
| Purity by SE-UPLC (%) | a. 99 | a. 99 | a. 99 | a. 99 | a. 99 | a. 99 | a. 99 | a. 99 | a. 99 | a. 99 | a. 99 |
| | b. 0 | b. 0 | b. 0 | b. 0 | b. 0 | b. 0 | b. 0 | b. 0 | b. 0 | b. 0 | b. 0 |
| | c. 1 | c. 1 | c. 1 | c. 1 | c. 1 | c. 1 | c. 1 | c. 1 | c. 1 | c. 1 | c. 1 |
| Charge Variant Analysis by | a. 30 | a. 30 | a. 31 | a. 30 | a. 30 | a. 30 | a. 32 | a. 31 | a. 33 | a. 32 | a. 31 |
| iCIEF (%) | b. 62 | b. 61 | b. 60 | b. 60 | b. 61 | b. 61 | b. 57 | b. 58 | b. 55 | b. 56 | b. 58 |
| | c. 9 | c. 9 | c. 9 | c. 9 | c. 9 | c. 10 | c. 11 | c. 11 | c. 12 | c. 12 | c. 11 |
| Particulate Analysis by LO | a. 66 | NR | NR | a. 82 | NR | a. 52 | a. 84 | a. 43 | a. 107 | a. 101 | a. 238 |
| (#/container) | b. 0 | | | b. 0 | | b. 0 | b. 0 | b. 0 | b. 0 | b. 0 | b. 3 |
| Particulate Analysis by MFI | NR | NR | NR | 36420 | NR | 27028 | 26024 | 45022 | 36308 | 19868 | 36722 |
| Endotoxin | <0.001 | NR | NR | NR | NR | <0.001 | NR | <0.001 | NR | NR | NR |
| Sterility | Meets | NR | NR | NR | NR | Meets | NR | Meets | NR | NR | NR |
| CCI | NDD | NR | NR | NR | NR | NDD | NR | NDD | NR | NDD | NR |

^{a}Data from release testing
A₂₈₀, absorbance at 280 nm;
C2P1, Cell Line 2 Process 1;
CCI, container closure integrity;
EU, endotoxin unit;
HMW, high molecular weight;
iCIEF, imaged capillary isoelectric focusing;
LEFVP, liquid essentially free from visible particulates;
LMW, low molecular weight;
LO, light obscuration;
MCE, microchip capillary electrophoresis;
MFI, Micro-Flow Imaging ™;
MP, main peak;
NDD, no dye detected;
NGHC, non-glycosylated heavy chain;
Not > BY2;
NR, not required;
PGMP, partially glycosylated main peak;
Ph. Eur., European Pharmacopoeia;
SE, size-exclusion;
UPLC, ultra-performance liquid chromatography;
USP, United States Pharmacopeia

The DP solution contains 50 mg/mL mAb1, 10 mM histidine, pH 6.0, 10% (w/v) sucrose, 20 mM arginine hydrochloride, and 0.1% (w/v) polysorbate 80. The following can be concluded from the stability studies:
mAb1 DP is stable when stored at 2° C. to 8° C. for at least 42 months.
The main degradation pathways identified were the formation of HMW species and charge variants.

### Overages

There are no overages included in the formula; however, a slight overfill is included to ensure that the correct volume is withdrawn from the vial.

One vial of DP contains 16.5 mL of 50 mg/mL mAb1. An overfill of 0.5 mL is contained in each vial, which is sufficient for an accurate withdrawal of 16.0 mL (800 mg of mAb1) of DP from the vial.

This overfill is not designed to compensate for losses during manufacture, degradation during manufacture, degradation during storage (shelf life), or to extend the expiration date. This overage is solely intended to enable the accurate withdrawal of 16.0 mL.

### Example 5: 400 mg mAb1 Liquid Drug Product (DP) Research Stability

This report summarizes the research stability study for mAb1 liquid Drug Product (DP) (50 mg/mL mAb1, 10 mM L-histidine, 20 mM L-arginine hydrochloride, 10% (w/v) sucrose, 0.1% (w/v) polysorbate 80, pH 6.0) with 8.5 mL fill (and 8.0 mL withdrawal volume) in 10R Type 1 borosilicate glass vials.

The DP was incubated under storage, accelerated, and stress conditions. The accelerated and stress conditions were selected to simulate the conditions beyond which the DP will not be subjected during manufacturing and handling, and to elucidate the degradation pathways for mAb1 DP.

The study was staged in the upright orientation under the conditions described below. The following stress/storage conditions and the duration of time points were examined in the mAb1 research stability studies.
Long term storage condition: 2-8° C., up to 60 months
Thermal accelerated condition: 25° C./60% RH, up to 6 months
Thermal stressed condition: 40° C./75% RH, up to 3 months
Physical stressed condition: agitation by vortexing at 1000 RPM for up to 120 minutes
Physical stressed condition: freezing and thawing at −30° C. up to 4 cycles

The following methods were used to analyze the stability of mAb1:
Color and appearance
pH
Protein concentration by Solo VPE
Purity by SE-UPLC
Charge Heterogeneity by iCIEF
Purity by MCE (reduced and non-reducing)—All timepoints, except only T_{end} (last timepoint) for agitation and F/T
Particulate Matter by HIAC
   0, 3, 6 m & annual TP (timepoint) for 5° C.
   3 m & T_{end} for 25° C./60% RH and 40° C./75% RH
   T_{end} for agitation and F/T
Particulate Matter by MFI
   0, 6 m & annual TP for 5° C.
   3 m & T_{end} for 25° C./60% RH and 40° C./75% RH
   T_{end} for agitation and F/T
Polysorbate 80 Concentration by CAD-UPLC
   0, 6 m, and annual TP for 5° C.
   T_{end} for 25° C./60% RH and 40° C./75% RH
   T_{end} for agitation and F/T
Potency by Bioassay—0, 6 m & annual TP for 5° C.

### Long-Term Storage Stability Study Results

Twelve months of research stability data are available for the mAb1 DP. mAb1 DP was physically and chemically stable when stored at storage condition (2-8° C.) for at least 12 months. No appreciable changes in stability were detected in any of the monitored attributes at 2-8° C. as shown in Table 7. These results indicate that mAb1 DP is stable for at least 12 months at storage conditions.

### Thermal Accelerated and Stress Stability Study Results

Results from the analysis of the mAb1 DP after incubation under accelerated and stress thermal conditions are provided in Table 8.

No appreciable changes in the physical or chemical stability were detected after incubation at 25° C./60% RH for 1 month, indicating that the mAb1 DP can be exposed to room temperature for up to 1 month. After 6 months incubation at 25° C./60% RH, formation of Region 3 by iCIEF was detected as illustrated in Table 9. See also Table 8.

After incubation for 3 months at 40° C./75% RH, appreciable formation of HMW species by SE-UPLC, LMW by MCE, and charge variants by iCIEF (illustrated in Table 10). For details on the charge variants at stress conditions, see Table 8. The results from incubation under accelerated and stress conditions demonstrated that an increase in HMW species, LMW species and formation of charge variants were the main degradation pathways for mAb1 DP.

TABLE 7
Stability of mAb1 Liquid Drug Product Stored at 2-8° C.

| | |
|---|---|
| Formulation | 50 mg/mL mAb1, 10 mM L-histidine, 20 mM L-arginine |
| | hydrochloride, 10% (w/v) sucrose, 0.1% (w/v) polysorbate |
| | 80, pH 6.0 |
| Container Closure | 10R Type 1 borosilicate glass vial with 20 mm gray |
| | chlorobutyl rubber stopper with FluroTec |
| Fill Volume | 8.5 mL |

| | | Length of Storage (months) | | | | | |
|---|---|---|---|---|---|---|---|
| Assay | | t = 0 | 1 | 3 | 6 | 9 | 12 |
| Color and Appearance^{a} | | Pass | Pass | Pass | Pass | Pass | Pass |
| Turbidity (Increase in OD | | 0.00 | 0.00 | 0.00 | 0.01 | 0.01 | 0.00 |
| 405 nm) | | | | | | | |
| pH | | 6.0 | 6.1 | 6.0 | 6.0 | 6.0 | 6.0 |
| Total Protein (mg/mL) | | 47.6 | 48.3 | 47.5 | 49.0 | 50.2 | 48.1 |
| Potency (%) | | 104 | NR | NR | 111 | NR | 85 |
| Reduced | Purity | 97.3 | 96.3 | 96.8 | 98.0 | 96.9 | 97.3 |
| MCE (%) | (LC + HC) | | | | | | |
| | NGHC | 2.3 | 2.9 | 2.0 | 1.9 | 2.9 | 2.5 |
| | LMW | 0.5 | 0.0 | 1.0 | 0.0 | 0.0 | 0.1 |
| Non-reduced | Purity | 97.7 | 97.5 | 96.4 | 97.8 | 97.3 | 98.2 |
| MCE (%) | (NGMP + | | | | | | |
| | MP) | | | | | | |
| | LMW | 2.2 | 2.2 | 3.1 | 2.0 | 2.4 | 1.7 |
| | HMW | 0.1 | 0.3 | 0.5 | 0.2 | 0.3 | 0.2 |
| SE-UPLC | Purity | 99.5 | 99.4 | 99.4 | 99.3 | 99.3 | 99.3 |
| (%) | (MP + | | | | | | |
| | LMW1) | | | | | | |
| | LMW | 0.0 | 0.0 | 0.0 | 0.0 | 0.0 | 0.0 |
| | HMW | 0.5 | 0.6 | 0.6 | 0.7 | 0.7 | 0.7 |
| iCIEF (%) | Region 1 | 33.8 | 33.2 | 33.5 | 31.9 | 31.4 | 31.2 |
| | Region 2 | 56.7 | 57.0 | 56.3 | 57.1 | 57.6 | 57.6 |
| | Region 3 | 9.5 | 9.8 | 10.2 | 11.0 | 11.1 | 11.2 |
| Particulate | ≥10 μm | 6 | NR | 57 | 66 | NR | 94 |
| Analysis by | ≥25 μm | 0 | NR | 3 | 3 | NR | 9 |
| LO | | | | | | | |
| (#/container) | | | | | | | |
| Particulate | 2 μm ≤ | 17 | NR | NR | 629 | NR | 459 |
| Analysis by | x < 10 μm | | | | | | |
| MFI^{b} (#/mL) | ≥10 μm | 0 | NR | NR | 10 | NR | 20 |
| | ≥25 μm | 0 | NR | NR | 4 | NR | 4 |

| | | | | | | |
|---|---|---|---|---|---|---|
| Polysorbate 80 (% w/v) | 0.11 | NR. | NR | 0.11 | NR | 0.10 |

^{a}Pass if clear to slightly opalescent, essentially free from visible particulates, and colorless to pale.
^{b}Contribution from air bubbles minimized by filtering out particles with aspect ratio >0.85.
HMW, high molecular weight;
iCIEF, imaged capillary isoelectric focusing;
LEFVP, liquid essentially free from visible particulates;
LMW, low molecular weight;
LO, light obscuration;
MCE, microchip capillary electrophoresis;
MFI, Micro-Flow Imaging ™;
MP, main peak;
Not > BY2, not more intensely colored than Reference Solution BY2;
Not > IV, not more turbid than Reference Suspension IV;
NR, not required per protocol;
Ph. Eur., European Pharmacopeia;
SE, size exclusion;
UPLC, ultra performance liquid chromatography;
USP, United States Pharmacopeia

TABLE 8
Stability of mAb1 Liquid Drug Product Incubated at 25° C./60% RH and 40° C./75% RH

| | |
|---|---|
| Formulation | 50 mg/mL mAb1, 10 mM L-histidine, 20 mM L-arginine |
| | hydrochloride, 10% (w/v) sucrose, 0.1% (w/v) |
| | polysorbate 80, pH 6.0 |
| Container Closure | 10R Type 1 borosilicate glass vial with 20 mm gray |
| | chlorobutyl rubber stopper with FluroTec |
| Fill Volume | 8.5 mL |

| | | | Storage at 25° C./60% | | | | Storage at 40° C./75% | | | |
|---|---|---|---|---|---|---|---|---|---|---|
| | | | RH (months) | | | | RH (months) | | | |
| Assay | | t = 0 | 0.5 | 1 | 3 | 6 | 0.25 | 0.5 | 1 | 3 |
| Color and appearance | | Pass^{a} | Pass | Pass | Pass | Pass | Pass | Pass | Pass | Pass |
| Turbidity (Increase in | | 0.00 | 0.00 | 0.00 | 0.01 | 0.02 | 0.00 | 0.00 | 0.01 | 0.02 |
| OD 405 nm) | | | | | | | | | | |
| pH | | 6.0 | 6.0 | 6.0 | 6.0 | 6.0 | 6.0 | 6.0 | 6.0 | 6.0 |
| Total Protein (mg/mL) | | 47.6 | 48.3 | 47.8 | 49.0 | 48.2 | 48.7 | 48.5 | 48.9 | 48.4 |
| Potency (%) | | 104 | NR | NR | NR | 80 | NR | NR | NR | 88 |
| Reduced | Purity | 97.3 | 97.6 | 97.8 | 96.6 | 96.4 | 97.5 | 95.9 | 95.8 | 91.5 |
| MCE (%) | (LC + HC) | | | | | | | | | |
| | NGHC | 2.3 | 2.2 | 2.2 | 3.0 | 3.1 | 2.3 | 3.0 | 3.1 | 5.0 |
| | LMW | 0.5 | 0.0 | 0.0 | 0.2 | 0.4 | 0.3 | 0.3 | 1.0 | 3.2 |
| Non-reduced | Purity | 97.7 | 97.9 | 97.6 | 97.4 | 97.0 | 97.3 | 97.1 | 96.7 | 93.3 |
| MCE (%) | (NGMP + | | | | | | | | | |
| | MP) | | | | | | | | | |
| | LMW | 2.2 | 2.1 | 2.2 | 2.4 | 2.7 | 2.5 | 2.7 | 3.2 | 6.4 |
| | HMW | 0.1 | 0.0 | 0.2 | 0.2 | 0.3 | 0.3 | 0.2 | 0.2 | 0.3 |
| SE-UPLC | Purity | 99.5 | 99.4 | 99.3 | 99.2 | 99.2 | 99.3 | 99.1 | 98.5 | 95.7 |
| (%) | (MP + | | | | | | | | | |
| | LMW1) | | | | | | | | | |
| | LMW | 0.0 | 0.0 | 0.0 | 0.0 | 0.0 | 0.0 | 0.0 | 0.1 | 1.4 |
| | HMW | 0.5 | 0.6 | 0.7 | 0.8 | 0.8 | 0.7 | 0.9 | 1.4 | 2.9 |
| iCIEF (%) | Region 1 | 33.8 | 34.3 | 34.1 | 34.4 | 37.7 | 31.1 | 36.6 | 39.7 | 55.1 |
| | Region 2 | 56.7 | 54.8 | 54.1 | 51.4 | 46.2 | 55.7 | 48.1 | 41.3 | 25.0 |
| | Region 3 | 9.5 | 11.0 | 11.8 | 14.5 | 16.1 | 13.3 | 15.4 | 19.0 | 19.9 |
| Particulate | ≥10 μm | 6 | NR | NR | 179 | 88 | NR | NR | NR | 177 |
| Analysis by | ≥25 μm | 0 | NR | NR | 9 | 0 | NR | NR | NR | 0 |
| LO | | | | | | | | | | |
| (#/container) | | | | | | | | | | |
| Particulate | 2 μm ≤ | 17 | NR | NR | 150 | 477 | NR | NR | NR | 290 |
| Analysis by | x < 10 μm | | | | | | | | | |
| MFI^{b} (#/mL) | ≥10 μm | 0 | NR | NR | 7 | 12 | NR | NR | NR | 0 |
| | ≥25 μm | 0 | NR | NR | 0 | 0 | NR | NR | NR | 0 |

| | | | | | | | | | |
|---|---|---|---|---|---|---|---|---|---|
| Polysorbate 80 (% w/v) | 0.11 | NR | NR | NR | 0.11 | NR | NR | NR | 0.11 |

^{a}Pass if clear to slightly opalescent, essentially free from visible particulates, and colorless to pale.
^{b}Contribution from air bubbles minimized by filtering out particles with aspect ratio >0.85.
HMW, high molecular weight;
iCIEF, imaged capillary isoelectric focusing;
LEFVP, liquid essentially free from visible particulates;
LMW, low molecular weight;
LO, light obscuration;
MCE, microchip capillary electrophoresis;
MFI, Micro-Flow Imaging ™;
MP, main peak;
Not > BY2, not more intensely colored than Reference Solution BY2;
Not > IV, not more turbid than Reference Suspension IV;
NR, not required per protocol;
Ph. Eur., European Pharmacopeia;
SE, size exclusion;
UPLC, ultra performance liquid chromatography;
USP, United States Pharmacopeia

TABLE 9
Summary of Change in Stability of mAb1 DP (400 mg)
after Incubation at 25° C./60% RH for 6 Months

| | | | Change in % | Change in % |
|---|---|---|---|---|
| | | | Region 1 | Region 3 |
| | Change in % | Change in % | (Acidic | (Basic |
| | HMW by SE- | LMW by | Region) by | Region) by |
| mAb1 DP | UPLC | Reduced MCE | iCIEF | iCIEF |
| 400 mg | +0.3 | +0.2 | +0.6 | +5.0 |

TABLE 10
Summary of Change in Stability of mAb1 DP (400 mg)
after Incubation at 40° C./75% RH for 3 Months

| | | | Change in % | Change in % |
|---|---|---|---|---|
| | | | Region 1 | Region 3 |
| | Change in % | Change in % | (Acidic | (Basic |
| | HMW by SE- | LMW by Non- | Region) by | Region) by |
| mAb1 DP | UPLC | reduced MCE | iCIEF | iCIEF |
| 400 mg | +2.4 | +4.2 | +21.3 | +10.4 |

### Agitation and Freezing and Thawing Stability Study Results

Results from the analysis of mAb1 DP after stress conditions of vortexing and freezing and thawing are provided in Table 11. No appreciable changes in the physical or chemical stability were detected after 120 minutes of vortex agitation and after 4 cycles of freezing (−30° C.) and thawing (room temperature). These results indicate that the mAb1 DP (400 mg) is stable to agitation stress and up to 4 cycles of freezing and thawing.

TABLE 11
Stability of mAb1 Liquid Drug Product - Effect of Agitation and Freeze/Thaw

| | |
|---|---|
| Formulation | 50 mg/mL mAb1, pH 6.0, 10 mM L-histidine, 20 mM |
| | L-arginine hydrochloride, 10% (w/v) sucrose, and |
| | 0.1% (w/v) polysorbate 80 |
| Container Closure | 10R Type 1 borosilicate glass vial with 20 mm gray |
| | chlorobutyl rubber stopper with FluroTec |
| Fill Volume | 8.5 mL |

| | | | Agitation | | Freezing/Thawing | |
|---|---|---|---|---|---|---|
| | | | (minutes) | | (cycles) | |
| Assay | | t = 0 | 60 | 120 | 2 | 4 |
| Color and Appearance | | Pass^{a} | Pass | Pass | Pass | Pass |
| Turbidity (Increase in OD | | 0.00 | 0.00 | 0.00 | 0.00 | 0.00 |
| 405 nm) | | | | | | |
| pH | | 6.0 | 6.0 | 6.0 | 6.0 | 6.0 |
| Total Protein (mg/mL) | | 47.6 | 49.4 | 49.6 | 48.8 | 48.7 |
| Potency (%) | | 104 | NR | 89 | 95 | NR |
| Reduced MCE | Purity | 97.3 | NF | 97.2 | NR | 96.2 |
| (%) | (LC + HC) | | | | | |
| | NGHC | 2.3 | NR | 1.8 | NR | 1.9 |
| | LMW | 0.5 | NR | 0.7 | NR | 1.7 |
| Non-reduced | Purity | 97.7 | NR | 97.7 | NR | 96.3 |
| MCE (%) | (NGMP + | | | | | |
| | MP) | | | | | |
| | LMW | 2.2 | NR | 2.1 | NR | 3.5 |
| | HMW | 0.1 | NR | 0.1 | NR | 0.2 |
| SE-UPLC (%) | Purity | 99.5 | 99.5 | 99.5 | 99.5 | 99.5 |
| | (MP + | | | | | |
| | LMW1) | | | | | |
| | LMW | 0.0 | 0.0 | 0.1 | 0.0 | 0.0 |
| | HMW | 0.5 | 0.5 | 0.5 | 0.5 | 0.5 |
| iCIEF (%) | Region 1 | 33.8 | NR | 33.0 | NR | 34.5 |
| | Region 2 | 56.7 | NR | 57.6 | NR | 56.4 |
| | Region 3 | 9.5 | NR | 9.3 | NR | 9.0 |
| Particulate | ≥10 μm | 6 | NR | 34 | NR | 9 |
| Analysis by LO | ≥25 μm | 0 | NF | 0 | NR | 0 |
| (#/container) | | | | | | |
| Particulate | 2 μm ≤ | 17 | NR | 308 | NR | 69 |
| Analysis by | x < 10 μm | | | | | |
| MFI^{b} (#/mL) | ≥10 μm | 0 | NR | 5 | NR | 0 |
| | ≥25 μm | 0 | NR | 0 | NR | 0 |

| | | | | | |
|---|---|---|---|---|---|
| Polysorbate 80 (% w/v) | 0.11 | NR | 0.11 | NR | 0.11 |

^{a}Pass if clear to slightly opalescent, essentially free from visible particulates, and colorless to pale yellow.
^{b}Contribution from air bubbles minimized by filtering out particles with aspect ratio >0.85.
HMW, high molecular weight;
iCIEF, imaged capillary isoelectric focusing;
LEFVP, liquid essentially free from visible particulates;
LMW, low molecular weight;
LO, light obscuration;
MCE, microchip capillary electrophoresis;
MFI, Micro-Flow Imaging ™;
MP, main peak;
Not > BY2, not more intensely colored than Reference Solution BY2;
Not > IV, not more turbid than Reference Suspension IV;
NR, not required per protocol;
Ph. Eur., European Pharmacopeia;
SE, size exclusion;
UPLC, ultra performance liquid chromatography;
USP, United States Pharmacopeia

### Conclusions

At the time of this report, 12 months of stability data are available from the stability study of mAb1 (400 mg presentation). This research stability study evaluated mAb1 DP stability under long-term, accelerated, and stress conditions. No appreciable changes in mAb1 DP stability are detected during long-term storage at 2-8° C. for up to 12 months in any of the monitored attributes. In addition, mAb1 DP was physically and chemically stable when subjected to agitation stress by vortex and freeze-thaw.

Results from this study support the recommended long-term storage condition of 2-8° C. for the mAb1 DP. The DP stability data supports storage at accelerated condition at up to 25° C./60% RH up to 1 month.

### Example 6: 50 mg/mL mAb1 Liquid Drug Product (DP) Formulation Robustness Study

The following formulation robustness studies were performed on mAb1 as a liquid drug product (DP). The liquid DP contains 50 mg/mL mAb1 in 10 mM L-histidine, 10% (w/v) sucrose, 0.1% (w/v) polysorbate 80, 20 mM L-arginine hydrochloride, pH 6.0 and stored in 20 mL Type 1 borosilicate glass vials. Because variations in any of these formulation factors could potentially impact the stability of the DP, a formulation robustness study was conducted to assess whether variations in the DP formulation composition, within the defined Proven Acceptable Range (PAR) would meaningfully affect the long-term stability of mAb1 DP.

All formulations were prepared and filled into 20 mL Type 1 glass vials with a 16.5 mL fill volume and assessed for long-term storage stability at 2-8° C. The formulation parameters and their ranges examined in this study are summarized in Table 12 and the formulations examined in the robustness study are shown in Table 13. The storage conditions for this study are shown in Table 14.

TABLE 12
Summary of Formulation Parameters and Formulation Ranges

| Formulation | Nominal | | Targeted PAR | | Range | |
|---|---|---|---|---|---|---|
| Factor | Level | | Range | | Tested | |
| mAb1 concentration | 50 | mg/mL | 45-55 | mg/mL | 45-55 | mg/mL |
| Sucrose concentration | 10% | (w/v) | 8-12% | (w/v) | 8-12% | (w/v) |
| L-histidine concentration | 10 | mM | 8-12 | mM | 8-12 | mM |
| L-arginine hydrochloride | 20 | mM | N/A | | Fixed | |
| concentration | | | | | | |
| Polysorbate 80 | 0.1% | (w/v) | 0.05%-0.2% | (w/v) | 0.05%-0.2% | (w/v) |

| | | | |
|---|---|---|---|
| concentration | | | |
| pH | 6.0 | 5.7-6.3 | 5.7-6.3 |

PAR, proven acceptable range

TABLE 13
Formulations Tested in the PAR Study

| | | | | L-arginine | | |
|---|---|---|---|---|---|---|
| | mAb1 | Sucrose | L-histidine | hydrochloride | Polysorbate | |
| | Concentration | Concentration | Concentration | Concentration | 80 | |
| Run # | (mg/mL) | % (w/v) | (mM) | (mM) | % (w/v) | pH |
| F1 | 45 | 12 | 12 | 20 | 0.05 | 6.0 |
| F2 | 55 | 8 | 8 | 20 | 0.05 | 6.0 |
| F3 | 55 | 8 | 12 | 20 | 0.2 | 5.7 |
| F4 | 45 | 12 | 12 | 20 | 0.2 | 6.3 |
| F5 | 45 | 8 | 8 | 20 | 0.2 | 6.0 |
| F6 | 45 | 12 | 8 | 20 | 0.2 | 5.7 |
| F7 | 55 | 8 | 12 | 20 | 0.05 | 6.3 |
| F8 | 55 | 12 | 12 | 20 | 0.05 | 6.3 |
| F9 | 55 | 8 | 8 | 20 | 0.2 | 6.3 |
| F10 | 55 | 12 | 8 | 20 | 0.05 | 5.7 |
| F11 | 45 | 12 | 8 | 20 | 0.05 | 5.7 |
| F12 | 45 | 8 | 8 | 20 | 0.05 | 6.3 |
| F13 | 45 | 8 | 12 | 20 | 0.05 | 5.7 |
| F14 | 55 | 12 | 12 | 20 | 0.2 | 6.0 |

TABLE 14
Study Conditions
DP Storage, Accelerated, and Stress (Thermal) Stability

| Storage Temperature | Length of Storage (months) |
|---|---|
| 5° C. (2-8° C.) | 0, 1, 3, 6, 9, and 12 |
| 25° C./60% RH | 0, 1, 3, and 6 |
| 40° C./75% RH | 0, 0.5, 1, and 3 |

DP Stress (Non-Thermal) Stability

| | |
|---|---|
| Agitation^{a} | 0 and 48 hours |

^{a}250 RPM on orbital shaker

The analytical testing plan utilized the following methods to assess stability of the formulations in Table 13:
osmolality by vapor pressure
viscosity
color and appearance
pH
protein content by Solo VPE
purity content by SEC
purity by MOE (non-reduced and reduced)
   FDS: t=0, −30° C. and −20° C. at 6 and 12 m
   DP: t=0, 50C at 6, and 12 m, 25° C./60% RH at 6 m, 40° C./75% RH at
   3 m, and agitation at the last time point
charge heterogeneity by iCIEF
   FDS: t=0, −30° C. and −20° C. at 3, 6, and 12 m
   DP: t=0, 50C at 3, 6, and 12 m, 25° C./60% RH at 1 and 6 m,
   40° C./75% RH at 3 m, and agitation at the last time point
polysorbate by CAD-UPLC
   FDS: t=0, −30° C. and −20° C. at 3, 6, and 12 m
   DP: t=0, 50C at 3, 6, and 12 m, 25° C./60% RH at 6 m, 40° C./75% RH at 3 m, and agitation at the last time point
particulate matter by MFI
   FDS: t=0, −30° C. and −20° C. at 3, 6, and 12 m
DP: t=0, 50C at 3, 6, and 12 m, 25° C./60% RH at 6 m, 40° C./75% RH at 3 m, and agitation at the last time point
particulate matter by HIAC
   DP: t=0, 50C at 3, 6, and 12 m, 25° C./60% RH at 6 m, 40° C./75% RH at 3 m, and agitation at the last time point.

### Long-term DP Stability Study Results

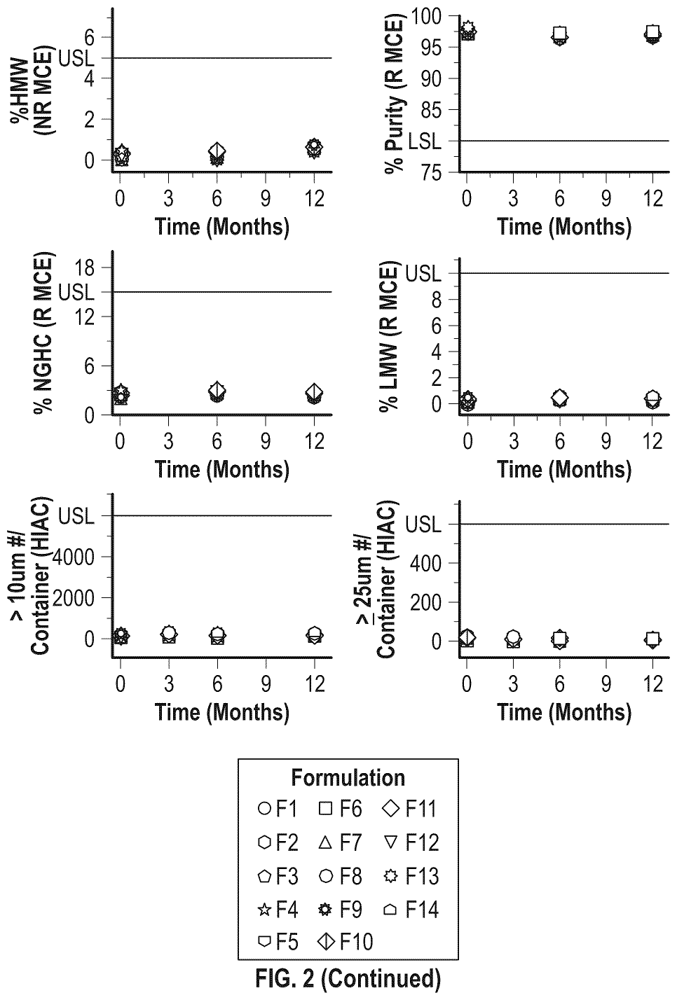

mAb1 DP formulations listed in Table 15 were stored at 2-8° C. The effects of varying protein concentration, sucrose concentration, pH, histidine concentration, and polysorbate 80 concentration on the long-term stability of mAb1 DP were examined. The quality attributes assessed were formation of molecular weight variants by SE-UPLC and MCE, formation of charge variants by iCIEF, appearance, pH, protein recovery, and subvisible particulate levels by HIAC and MFI. The results illustrating stability at 2-8° C. storage over 12 months are shown in FIG. 2.

These results indicate that after 12 months of storage at 2-8° C., varying the formulation compositions within the ranges tested resulted in no meaningful impact to the stability or quality of mAb1.
No change in color, precipitate or visible particulate was detected by visual inspection.
No meaningful changes in protein recovery were observed by Solo VPE.
The pH of the formulations was stable.
No meaningful increases in subvisible particulates were observed by HIAC.
   All values were below the acceptable limits set by USP <787>.
No meaningful changes were observed in the levels of HMW species or LMW species as determined by SE-UPLC or MCE.
No meaningful differences were observed in the levels of acidic (Region 1) or basic (Region 3) charge variants as determined by iCIEF.

The data demonstrate that the 50 mg/mL mAb1 DP formulation (800 mg DP presentation) is robust with respect to all the assessed quality attributes within the tested formulation composition range.

### Accelerated and Stress (Thermal) DP Stability Study Results

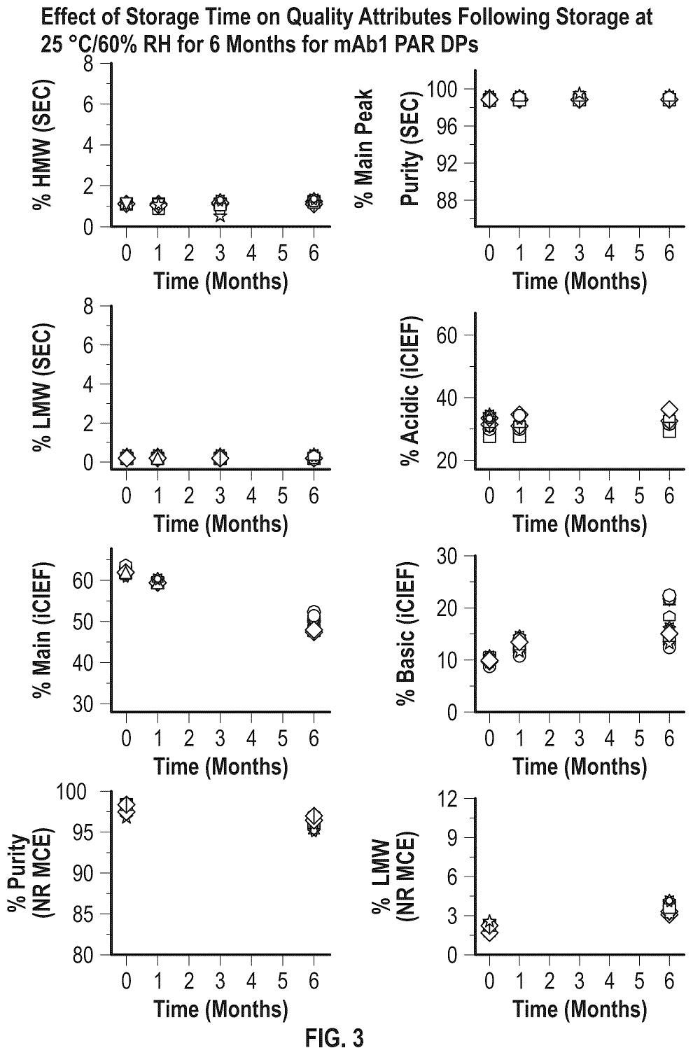

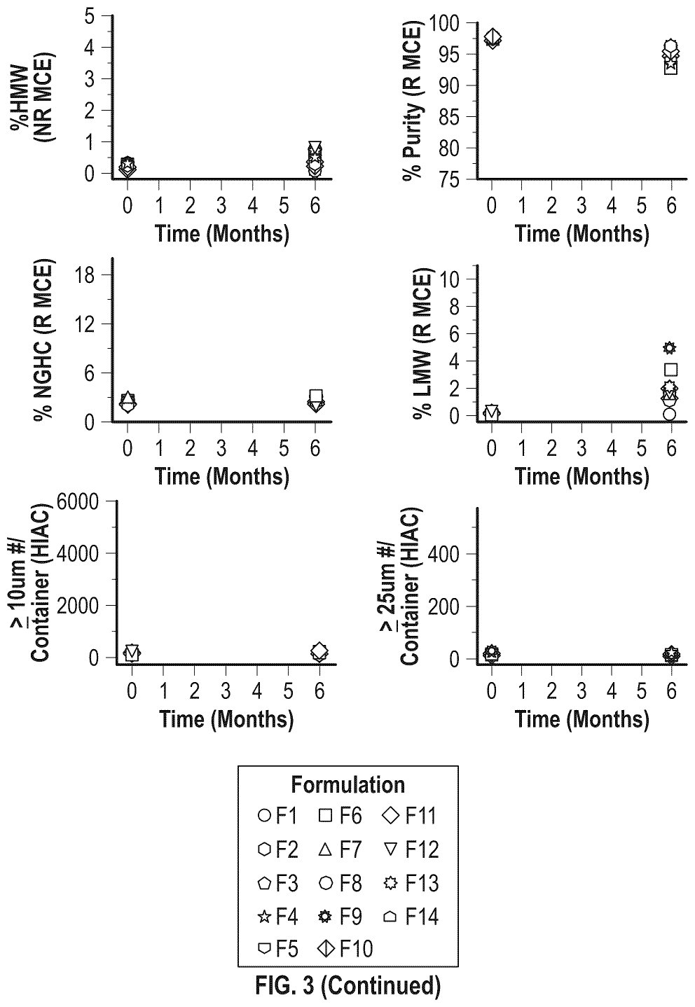

The results for illustrating stability over time at 25° C./60% RH (accelerated and stress thermal stability study) are summarized in FIG. 3.

No meaningful change in stability was observed when formulations were incubated for one month at 25° C./60% RH, indicating that the 50 mg/mL C2P1 DP formulation (800 mg presentation) within the tested formulation composition range can be exposed to short periods of time at room temperature. Data not shown.

After incubation for 3 months at 40° C./75% RH, appreciable formation of HMW, LMW, and charge variants were detected. The results from the accelerated conditions demonstrated that the formation of HMW, LMW, and charge variants were the main degradation pathway for the 50 mg/mL mAb1 DP within the tested formulation composition range. Data not shown.

### Agitation DP Stability Study Results

Among the 14 formulations examined in the PAR stress (non-thermal) stability study, all formulations were physically and chemically stable when agitated (250 RPM on orbital-shaking at ambient temperature) for 48 hours.

### Osmolality and Viscosity Results

The osmolality and viscosity of the PAR formulations described in Table 13 were measured and summarized in Table 15. Viscosity ranged from about 2.0 CP to about 3.0 CP at 20° C. Osmolality ranged from about 330 to about 530 mOsm/kg.

TABLE 15
Osmolality and Viscosity of mAb1 Formulation Drug Product

| | | | | L- | | | | |
|---|---|---|---|---|---|---|---|---|
| | mAb1 | Polysorbate | L-arginine | Histidine | Sucrose | | Osmolality | Viscosity |
| | Conc. | 80 Conc. | HCl Conc. | Conc. | Conc. | | (mOsm/ | at 20° C. |
| F# | (mg/mL) | (% w/v) | (mM) | (mM) | (% w/v) | pH | kg) | (cP) |
| F1 | 45 | 0.05 | 20 | 12 | 12 | 6.0 | 514 | 2.27 |
| F2 | 55 | 0.05 | 20 | 8 | 8 | 6.0 | 339 | 2.85 |
| F3 | 55 | 0.2 | 20 | 12 | 8 | 5.7 | 347 | 2.50 |
| F4 | 45 | 0.2 | 20 | 12 | 12 | 6.3 | 503 | 2.45 |
| F5 | 45 | 0.2 | 20 | 8 | 8 | 6.0 | 348 | 2.04 |
| F6 | 45 | 0.2 | 20 | 8 | 12 | 5.7 | 510 | 2.41 |
| F7 | 55 | 0.05 | 20 | 12 | 8 | 6.3 | 408 | 2.44 |
| F8 | 55 | 0.05 | 20 | 12 | 12 | 6.3 | 504 | 2.71 |
| F9 | 55 | 0.2 | 20 | 8 | 8 | 6.3 | 346 | 2.45 |
| F10 | 55 | 0.05 | 20 | 8 | 12 | 5.7 | 493 | 2.65 |
| F11 | 45 | 0.05 | 20 | 8 | 12 | 5.7 | 496 | 2.41 |
| F12 | 45 | 0.05 | 20 | 8 | 8 | 6.3 | 338 | 2.15 |
| F13 | 45 | 0.05 | 20 | 12 | 8 | 5.7 | 342 | 2.04 |
| F14 | 55 | 0.2 | 20 | 12 | 12 | 6.0 | 519 | 2.78 |

### FDS Stability Results

The bulk FDS used to produce mAb1 PAR formulation DP were exposed to 2 freeze/thaw cycles (freezing at −30° C. and thawing at room temperature) prior to the DP fill. The bulk FDS were physically and chemically stable when subjected to 2 freeze/thaw cycles. Data not shown.

mAb1 PAR formulation stored in polycarbonate vials were physically and chemically stable when stored for up to 12 months at −20° C. or −30° C. Data not shown.

### Conclusions

The formulation robustness study demonstrated that variations in the composition of the mAb1 formulation within the ranges studied will not adversely impact the stability or quality of the 50 mg/mL mAb1 DP under the recommended storage condition (2-8° C.). Therefore, the 50 mg/mL mAb1 DP formulation is considered robust for protein concentration ranging from 45 mg/mL to 55 mg/mL, sucrose concentration ranging from 8% to 12% (w/v), L-histidine concentration ranging from 8 mM to 12 mM, polysorbate 80 concentration ranging from 0.05% to 0.2% (w/v), at 20 mM L-arginine hydrochloride, and pH ranging from 5.7 to 6.3.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 16
Informal Sequence Listing

| SEQ | | |
|---|---|---|
| ID NO. | Sequence | Description |
| 1. | QVQLVESGGG VVQPGRSLRL SCVASGFTFS SYGMHWVRQA | AA amino |
| | PGKGLEWVAI IWYDGSNKYY ADSVKGRFTI SRDNSKNTQY | acid |
| | LQMNSLRAED TAVYYCASVA TSGDFDYYGM DVWGQGTTVT | sequence |
| | VSS | |
| 2. | EIVLTQSPAT LSLSPGERTT LSCRASQRIS TYLAWYQQKP | AA amino |
| | GQAPRLLIYD ASKRATGIPA RFSGSGSGTG FTLTISSLEP | acid |
| | EDFAVYYCQQ RSNWPLTFGG GTKVEIK | sequence |
| 3. | GFTFSSYG | AA amino |
| | | acid |
| | | sequence |
| 4. | IWYDGSNK | AA amino |
| | | acid |
| | | sequence |
| 5. | ASVATSGDFD YYGMDV | AA amino |
| | | acid |
| | | sequence |
| 6. | QRISTY | AA amino |
| | | acid |
| | | sequence |
| 7. | DAS | AA amino |
| | | acid |
| | | sequence |
| 8. | QQRSNWPLT | AA amino |
| | | acid |
| | | sequence |
| 9. | QVQLVESGGG VVQPGRSLRL SCVASGFTFS SYGMHWVRQA | AA amino |
| | PGKGLEWVAI IWYDGSNKYY ADSVKGRFTI SRDNSKNTQY | acid |
| | LQMNSLRAED TAVYYCASVA TSGDFDYYGM DVWGQGTTVT | sequence |
| | VSSASTKGPS VFPLAPCSRS TSESTAALGC LVKDYFPEPV | |
| | TVSWNSGALT SGVHTFPAVL QSSGLYSLSS VVTVPSSSLG | |
| | TKTYTCNVDH KPSNTKVDKR VESKYGPPCP PCPAPPVAGP | |
| | SVFLFPPKPK DTLMISRTPE VTCVVVDVSQ EDPEVQFNWY | |
| | VDGVEVHNAK TKPREEQFNS TYRVVSVLTV LHQDWLNGKE | |
| | YKCKVSNKGL PSSIEKTISK AKGQPREPQV YTLPPSQEEM | |
| | TKNQVSLTCL VKGFYPSDIA VEWESNGQPE NNYKTTPPVL | |
| | DSDGSFFLYS RLTVDKSRWQ EGNVFSCSVM HEALHNHYTQ | |
| | KSLSLSLGK | |
| 10. | EIVLTQSPAT LSLSPGERTT LSCRASQRIS TYLAWYQQKP | AA amino |
| | GQAPRLLIYD ASKRATGIPA RFSGSGSGTG FTLTISSLEP | acid |
| | EDFAVYYCQQ RSNWPLTFGG GTKVEIKRTV AAPSVFIFPP | sequence |
| | SDEQLKSGTA SVVCLLNNFY PREAKVQWKV DNALQSGNSQ | |
| | ESVTEQDSKD STYSLSSTLT LSKADYEKHK VYACEVTHQG | |
| | LSSPVTKSFN RGEC | |

1. A liquid pharmaceutical formulation comprising:
(a) an antibody which binds specifically to human lymphocyte activation gene-3 (LAG-3), wherein the antibody comprises three heavy chain complementarity determining regions (CDRs) (HCDR1, HCDR2 and HCDR3) contained in a heavy chain variable region (HCVR) of SEQ ID NO: 1 and three light chain CDRs (LCDR1, LCDR2 and LCDR3) contained in a light chain variable region (LCVR) of SEQ ID NO: 2;
(b) a buffer comprising histidine, acetate, or phosphate;
(c) an organic cosolvent comprising polysorbate or polyethylene glycol; and
(d) a stabilizer comprising a sugar or an amino acid;
wherein the formulation has a pH of 6.0±0.3.

2. The pharmaceutical formulation of claim 1, wherein the antibody concentration is from 5 mg/mL±0.75 mg/mL to 250 mg/mL±37.5 mg/mL.

3. The pharmaceutical formulation of claim 2, wherein the antibody concentration is 50 mg/mL±7.5 mg/mL.

4. The pharmaceutical formulation of claim 2, wherein the antibody concentration is 100 mg/mL±15 mg/mL.

5. The pharmaceutical formulation of claim 2, wherein the antibody concentration is 150 mg/mL±22.5 mg/mL.

6. The pharmaceutical formulation of claim 2, wherein the antibody concentration is 175 mg/mL±26.25 mg/mL.

7. The pharmaceutical formulation of claim 1, wherein the buffer is histidine and the histidine buffer concentration is from 5 mM±1 mM to 20 mM±4 mM.

8. The pharmaceutical formulation of claim 7, wherein the histidine buffer concentration is 10 mM±2 mM.

9. The pharmaceutical formulation of claim 1, wherein the cosolvent is polysorbate and the polysorbate concentration is from 0.01%±0.005% to 0.5%±0.25% w/v.

10. (canceled)

11. The pharmaceutical formulation of claim 9, wherein the cosolvent is polysorbate and the polysorbate concentration is 0.2%±0.1% w/v.

12. The pharmaceutical formulation of claim 1, wherein the cosolvent is polysorbate and the cosolvent is polysorbate 80.

13. The pharmaceutical formulation of claim 1, wherein the stabilizer comprises sucrose and the sucrose concentration is from 1% to 20%±4% w/v.

14. (canceled)

15. The pharmaceutical formulation of claim 13, wherein the sucrose concentration is 10%±2% w/v.

16. The pharmaceutical formulation of claim 1, wherein the stabilizer comprises a sugar and an amino acid.

17. The pharmaceutical formulation of claim 16, wherein the amino acid is arginine hydrochloride at a concentration of from 0 to 80 mM±16 mM.

18. The pharmaceutical formulation of claim 17, wherein the arginine hydrochloride concentration is 20 mM±4 mM.

19. The pharmaceutical formulation of claim 1, comprising:
(a) 175 mg/mL±26.25 mg/mL antibody,
(b) of from 5 mM±1 mM to 20 mM±4 mM histidine buffer,
(c) of from 0.1%±0.05% to 0.5%±0.25% w/v polysorbate,
(d) of from 1%±0.2% to 15%±3% w/v sucrose, and
(e) of from 1 mM±0.2 mM to 100 mM±20 mM arginine hydrochloride,
at pH 6.0±0.3.

20. The pharmaceutical formulation of claim 1, comprising:
(a) 150 mg/mL±22.5 mg/mL antibody,
(b) of from 5 mM±1 mM to 20 mM±4 mM histidine buffer,
(c) of from 0.1%±0.05% to 0.5%±0.25% w/v polysorbate,
(d) of from 1%±0.2% to 10%±2% w/v sucrose, and
(e) of from 1 mM±0.2 mM to 100 mM±20 mM arginine hydrochloride;
at pH 6.0±0.3.

21. The pharmaceutical formulation of claim 1, comprising:
(a) 100 mg/mL±15 mg/mL antibody,
(b) of from 5 mM±1 mM to 20 mM±4 mM histidine buffer,
(c) of from 0.1%±0.05% to 0.5%±0.25% w/v polysorbate,
(d) of from 1%±0.2% to 10%±2% w/v sucrose, and
(e) of from 1 mM±0.2 mM to 100 mM±20 mM arginine hydrochloride;
at pH 6.0±0.3.

22. The pharmaceutical formulation of claim 21, comprising:
(a) 100 mg/mL±15 mg/mL antibody,
(b) 10 mM±2 mM histidine buffer,
(c) 0.1%±0.05% w/v polysorbate,
(d) 10%±2% w/v sucrose, and
(e) 20 mM±4 mM arginine hydrochloride,
at pH 6.0±0.3.

23. The pharmaceutical formulation of claim 1, comprising:
(a) 50 mg/mL±7.5 mg/mL antibody,
(b) of from 5 mM±1 mM to 20 mM±4 mM histidine buffer,
(c) of from 0.1%±0.05% to 0.5%±0.25% w/v polysorbate,
(d) of from 1%±0.2% to 10%±2% w/v sucrose, and
(e) of from 0 mM to 80 mM±16 mM arginine hydrochloride,
at pH 6.0±0.3.

24. The pharmaceutical formulation of claim 23, comprising:
(a) 50 mg/mL±7.5 mg/mL antibody,
(b) 10 mM±2 mM histidine buffer,
(c) 0.1%±0.05% w/v polysorbate,
(d) 5%±1% w/v sucrose, and
(e) 20 mM±4 mM arginine hydrochloride,
at pH 6.0±0.3.

25. The pharmaceutical formulation of claim 23, comprising:
(a) 50 mg/mL±7.5 mg/mL antibody,
(b) 10 mM±2 mM histidine buffer,
(c) 0.1%±0.05% w/v polysorbate,
(d) 10%±2% w/v sucrose, and
(e) 20 mM±4 mM arginine hydrochloride,
at pH 6.0±0.3.

26. The pharmaceutical formulation of claim 23, comprising:
(a) 50 mg/mL±7.5 mg/mL antibody,
(b) 10 mM±2 mM histidine buffer,
(c) 0.1%±0.05% w/v polysorbate, and
(d) 5%±1% w/v sucrose, and
at pH 6.0±0.3.

27. The pharmaceutical formulation of claim 1, wherein:
at least 90% of the antibody has native conformation after 28 days at 40° C.;
at least 45% of the antibody is the main charge variant of the antibody after 28 days at 40° C.;
at least 98% of the antibody has native conformation after three months at 25° C.;
at least 50% of the antibody is the main charge variant of the antibody after three months at 25° C.;
at least 98% of the antibody has native conformation after 12 months at 2-8° C.;
at least 50% of the antibody is the main charge variant of the antibody after 12 months at 2-8° C.;
at least 98% of the antibody has native conformation after 24 months at 2-8° C.; or
at least 50% of the antibody is the main charge variant of the antibody after 24 months at 2-8° C.

28.-40. (canceled)

41. A pharmaceutical formulation comprising:
(a) 50 mg/mL±7.5 mg/mL of an antibody that binds specifically to LAG-3, wherein the antibody comprises an HCVR of SEQ ID NO: 1 and an LCVR of SEQ ID NO: 2,
(b) 10 mM±2 mM histidine buffer, pH 6±0.3,
(c) 0.1%±0.05% w/v polysorbate 80,
(d) 10%±2% w/v sucrose, and
(e) 20 mM±4 mM arginine hydrochloride.

42. The pharmaceutical formulation of claim 41, wherein:
(i) ≥90% of the antibodies have a molecular weight of 145 kDa±1 kDa; and
(ii) more than 98% of the antibodies have native conformation upon storage for 12 months at 2-8° C.

43. The pharmaceutical formulation of claim 41 consisting of: (a) 50 mg/mL±7.5 mg/mL of the antibody, (b) 10.0 mM±2.0 mM histidine buffer, (c) 0.1%±0.05% w/v polysorbate 80, (d) 10%±2% w/v sucrose; and (e) 20 mM±4 mM arginine hydrochloride, in water at pH 6.0±0.3.

44. A pharmaceutical formulation comprising:
(a) 5-250 mg/ml of an antibody that binds specifically to LAG-3, wherein the antibody comprises an HCVR of SEQ ID NO: 1 and an LCVR of SEQ ID NO: 2,
(b) 10 mM±2 mM histidine buffer, pH 6.0±0.3,
(c) 0.1%±0.05% w/v polysorbate 80, and
(d) 5%±1% w/v sucrose.

45. The pharmaceutical formulation of claim 44 further comprising 1 mM±0.2 mM to 100 mM±20 mM arginine hydrochloride.

46. The pharmaceutical formulation of claim 1, wherein the antibody comprises a HCDR1 of SEQ ID NO: 3, a HCDR2 of SEQ ID NO: 4, a HCDR3 of SEQ ID NO: 5, a LCDR1 of SEQ ID NO: 6, a LCDR2 of SEQ ID NO: 7, and a LCDR3 of SEQ ID NO: 8.

47. The pharmaceutical formulation of claim 46, wherein the antibody comprises a HCVR of SEQ ID NO: 1 and a LCVR of SEQ ID NO: 2.

48. The pharmaceutical formulation of claim 1, wherein the antibody comprises a HCVR having 90% sequence identity to SEQ ID NO: 1.

49. The pharmaceutical formulation of claim 1, wherein the antibody comprises a LCVR having 90% sequence identity to SEQ ID NO: 2.

50. (canceled)

51. The pharmaceutical formulation of claim 1, wherein the antibody comprises a heavy chain and light chain, wherein the heavy chain comprises an amino acid sequence of SEQ ID NO: 9.

52. The pharmaceutical formulation of claim 1, wherein the antibody comprises a heavy chain and light chain, wherein the heavy chain comprises an amino acid sequence comprising amino acids 1-448 of SEQ ID NO: 9.

53. The pharmaceutical formulation of claim 1, wherein the antibody comprises a heavy chain and light chain, wherein the light chain comprises an amino acid sequence of SEQ ID NO: 10.

54. The pharmaceutical formulation of claim 1, wherein the antibody comprises a heavy chain/light chain comprising amino acid sequences of SEQ ID NOs: 9/10.

55. The pharmaceutical formulation of claim 1, wherein the antibody comprises a heavy chain amino acid sequence comprising amino acids 1-448 of SEQ ID NO: 9, and a light chain comprising an amino acid sequence of SEQ ID NO: 10.

56. A pharmaceutical formulation of claim 1, wherein said formulation is contained in a container.

57. The pharmaceutical formulation of claim 56, wherein the container is a vial.

58. The pharmaceutical formulation of claim 57, wherein the vial is 2 ml, 5 ml, 10 mL, or 20 ml Type 1 clear glass vial.

59. The pharmaceutical formulation of claim 57, wherein the container is a syringe.

60. The pharmaceutical formulation of claim 57, wherein the syringe is low-tungsten glass.

61. The pharmaceutical formulation of claim 57, wherein the container is a prefilled syringe.

62. The pharmaceutical formulation of claim 57, contained in an autoinjector.

63. A kit comprising a pharmaceutical formulation of claim 1, a container, and instructions.

64. The kit of claim 63, wherein the container is a glass vial.

65. The kit of claim 63, wherein the container is a prefilled syringe.

66. The kit of claim 63, wherein the container is an autoinjector.
